(19) United States
(12) Patent Application Publication
(10) Pub. No.: US 2020/0365232 A1
(43) Pub. Date: Nov. 19, 2020

(54) ADAPTIVE ORDER FULFILLMENT AND TRACKING METHODS AND SYSTEMS

(71) Applicant: Tempus Labs, Chicago, IL (US)

(72) Inventors: Charles Jaros, Chicago, IL (US); Robert Tell, Chicago, IL (US); Thomas Steinmetz, Chicago, IL (US); Elle Christina Moore, Chicago, IL (US); Isaiah D. Simpson, Chicago, IL (US)

(21) Appl. No.: 16/927,976

(22) Filed: Jul. 13, 2020

Related U.S. Application Data
(63) Continuation-in-part of application No. 16/771,451.
(60) Provisional application No. 62/873,693, filed on Jul. 12, 2019.
(60) Provisional application No. 62/746,997, filed on Oct. 17, 2018.

(51) Int. Cl. G16B 20/20; G16H 10/60; G16H 10/40; G16B 50/30; G16H 50/20; G16H 50/30

(52) U.S. Cl. CPC G16B 20/20; G16H 10/60; G16H 50/30; G16B 50/30; G16H 50/20; G16H 10/40

(57) ABSTRACT

A genomic test processing system and method employ an order management engine and one or more order processing engines, the order processing engines including a receiving engine, an execution engine, and a broadcasting engine. The receiving engine receives a state of an order from the order management engine. The execution engine determines a sequence of steps to advance the received state of an order to a final state, iteratively designates each step of the sequence of steps as completed before initiating the next step of the sequence of steps, and advances the state of the order to a final state when a last step of the sequence of steps is completed. The broadcasting engine broadcasts the final state of the order to the order management engine. The order management engine causes one of the order processing engines to generate a next-generation sequencing report from the final state of the order.

### CROSS-REFERENCE TO RELATED APPLICATIONS

This application claims the benefit of priority to U.S. provisional patent application No. 62/873,693, which was filed on Jul. 12, 2019, and which was titled “Adaptive Order Fulfillment and Tracking Methods and Systems.” The application also is a continuation-in-part of U.S. patent application Ser. No. 16/771,451, which was filed on Jun. 10, 2020, and which is titled “Data Based Cancer Research And Treatment Systems And Methods,” which is a U.S. national stage filing under 35 U.S.C. § 371 of international application No. PCT/US2019/056713, which was filed Oct. 17, 2019, and which is titled “Data Based Cancer Research and Treatment Systems and Methods,” which claims the benefit of priority to U.S. provisional patent application No. 62/746,997, which was filed on Oct. 17, 2018, and which is titled “Data Based Cancer Research And Treatment Systems And Methods.” This application also is a continuation-in-part of U.S. patent application Ser. No. 16/657,804, which was filed on Oct. 18, 2019, and which is titled “Data Based Cancer Research And Treatment Systems And Methods.” The contents of each of these applications is incorporated herein in their entirety by reference.

### STATEMENT REGARDING FEDERALLY SPONSORED RESEARCH OR DEVELOPMENT

N/A

### BACKGROUND OF THE DISCLOSURE

The field of the disclosure is complex medical testing order processing and management methods and systems and more specifically adaptive order processing systems for generating customized complex orders including items to be facilitated by many different system resources, managing those resources to complete order items and ultimately generate order reports and to enable visualization of real time and historical order status.

Hereafter, unless indicated otherwise, the following terms and phrases will be used as described. The term “physician” will be used to refer generally to any health care provider including but not limited to a primary care physician, a medical specialist, an oncologist, a psychiatrist, a nurse, a medical assistant, etc.

The phrase “cancer state” will be used to refer to a cancer patient's overall condition including diagnosed cancer, location of cancer, cancer stage, other cancer characteristics, other user conditions (e.g., age, gender, weight, race, genetics, habits (e.g., smoking, drinking, diet)), other pertinent medical conditions (e.g., high blood pressure, other diseases, etc.), medications, other pertinent medical history, current side effects of cancer treatments and other medications, etc.

The term “consume” will be used to refer to any type of consideration, use, or other activity related to any type of system data, tissue samples, etc., whether or not that consumption is exhaustive (e.g., used only once, as in the case of a tissue sample that cannot be reproduced) or persists for use by multiple entities (e.g., used multiple times as in the case of a simple data value).

The term “specialist” will be used to refer to any person other than the physician that operates within the disclosed systems to collect, develop, analyze or otherwise process system data, tissue samples or other information types (e.g., medical images) to generate any intermediate system work product or final work product where intermediate work product includes any data set, conclusions, tissue or other samples, grown tissues or samples, or other information for consumption by one or more other system specialists and where final work product includes data, conclusions or other information that is placed in a final or conclusory report for a system client. For instance, the phrase “abstractor specialist” will be used to refer to person that consumes data available in clinical records provided by a physician to generate normalized data for use by other system specialists, the phrase “sequencing specialist” will be used to refer to a person that consumes a tissue sample to generate DNA and/or RNA genomic data for use by other system specialists, the phrase “pathology specialist” will be used to refer to a scientist or physician specializing in pathology, etc.

The phrase “system entity” will be used to refer to any department, specialist, software application, etc., that performs any activity related to system data, tissue samples, or other system information. For instance, a genome sequencing lab and a radiology department are two examples of system entities. As another instance, an application program that receives radiology images and uses that data to generate a three dimensional representation of a tumor and surrounding tissue as well as the tumor's location and juxtaposition within the surrounding tissue is another system entity.

The phrase “deliverable consumer” will be used to refer to any system entity that consumes any system data, samples, or other information in any way including both specialists and software application programs that automatically consume data, samples, information or other deliverables independent of any initiating human activity.

The phrase “treatment planning” will be used to refer to an overall process that includes one or more sub-processes that process clinical and other data and samples (e.g., tumor tissue) to generate intermediate data deliverables and eventually final work product in the form of one or more final reports provided to clients. Thus, treatment planning may include data generation and processes used to generate that data as well as ultimate prescriptive plans for addressing a patient's ailments.

The phrases “Matched Tumor-Normal”, “Tumor-Normal Matched”, and “Tumor-Normal Sequencing” means processing genomic information from a subject's normal, non-cancerous, germline sample, such as saliva, blood, urine, stool, hair, healthy tissue, or other collections of cells or fluids from a subject, and genomic information from a subject's tumor, somatic sample, such as smears, biopsies or other collections of cells or fluids from a subject which contain tumor tissue, cells, or DNA (especially circulating tumor DNA, ctDNA). DNA and RNA features which have been identified from a next generation sequencing (NGS) of a subject's tumor or normal specimen may be cross referenced to remove genomic mutations and/or variants which appear as part of a subject's germline from the somatic analysis. The use of a somatic and germline dataset leads to substantial improvements in mutation identification and a reduction in false positive rates. “Tumor-Normal Matched Sequencing” provides a more accurate variant calling due to improved germline mutation filtering. For example, generating a somatic variant call based at least in part on the germline and somatic specimen may include identifying common mutations and removing them. In such a manner, variant calls from the germline are removed from variant calls from the somatic as non-driver mutations. A variant call that occurs in both the germline and the somatic specimen may be presumed to be normal to the patient and removed from further bioinformatic calculations.

The phrase “disease state” means a state of disease, such as cancer, cardiology, depression, mental health, diabetes, infectious disease, epilepsy, dermatology, autoimmune diseases, or other diseases. A disease state may reflect the presence or absence of disease in a subject, and when present may further reflect the severity of the disease.

Medical treatment prescriptions and treatment plans are typically based on an understanding of how treatments affect illness (e.g., treatment results) including how well specific treatments eradicate illness, duration of specific treatments, duration of healing processes associated with specific treatments and typical treatment specific side effects. Ideally treatments result in complete elimination of an illness in a short period with minimal or no adverse side effects. In some cases cost is also a consideration when selecting specific medical treatments for specific ailments.

Knowledge about treatment results is often based on analysis of empirical data developed over decades or even longer time periods during which physicians and/or researchers have recorded treatment results for many different patients and reviewed those results to identify generally successful ailment specific treatments. Researchers and physicians give medicine to patients or treat an ailment in some other fashion, observe results and, if the results are good, the researchers and physicians use the treatments again for similar ailments. If treatment results are bad, a researcher foregoes prescribing the associated treatment for a next encountered similar ailment and instead tries some other treatment. Treatment results are sometimes published in medical journals and/or periodicals so that many physicians can benefit from a treating physician's insights and treatment results.

Optimized cancer treatment planning, or precision medicine, for specific patients and cancer states is challenging for several reasons. First, more than most illnesses, time is of the essence when it comes to most cancer treatments where delay by just a few weeks or even days can have life and death consequences for an afflicted patient. Unfortunately, thorough and optimized cancer treatment planning is extremely complex requiring a series of activities by many specialists with different technical disciplines, all of which take time. In addition the various purposes of testing, including surveillance testing, screening testing, and diagnostic testing, can complicate the overall process for conducting testing. Surveillance testing may involve random sampling of a certain percentage of a specific population to monitor for increasing or decreasing prevalence and determining the population effect from community interventions. Screening testing involves looking for occurrence at the individual level even if there is no individual reason to suspect the patient has the condition or disease being screened for. This includes screening of individuals with the intent of making individual decisions based on the test results. Screening tests are intended to identify individuals prior to development of symptoms associated with the condition or disease, so that measures can be taken when patients who do screen positive (e.g. for cancer, to begin therapy for the patient; or for infectious disease, to prevent those individuals from infecting others). Diagnostic testing is also looking for occurrence of a condition or disease at the individual level and can be performed if there is a particular reason to suspect that an individual may have a condition or disease. Diagnostic tests in cancer, for instance, may be run in order to diagnose whether a lump found in a patient is benign or a tumor. Diagnostic tests in infection, for instance, may be run to diagnose an infection in patients suspected of infection by their healthcare provider such as in symptomatic individuals, individuals who have had a recent exposure, or individuals who are in a high-risk group with known exposure.

Second, there are more than 250 known cancer types and each type may be in one of first through fourth stages where, in each stage, the cancer may have many different characteristics so that the number of possible “cancer varieties” is relatively large which makes the sheer volume of knowledge required to fully comprehend all possible treatment results unwieldy and effectively inaccessible.

Third, for most cancer states, there are several different treatment options where each general option can be customized for a specific cancer state and patient condition. In many cases there are combinations of different treatment options which complicate the planning process even further. Understanding all treatment options and combinations for a specific case is a daunting task which is exacerbated over time as more treatment options and combinations of options are identified and developed.

Fourth, for some cancer states there are no accepted best treatment plan practices and, in these cases, physicians often have to turn to clinical studies to find treatment options for associated patients. Even in some cases where best treatment practices have been developed, one or more clinical trials may present better options for some cancer states given treatment results or other factors. Unfortunately there are hundreds and at times even thousands of clinical cancer studies being performed all the time where there are cancer state related qualifications as well as timing requirements for most of the studies. Diligently tracking all studies, timing and state qualifications is essentially impossible for any physician.

Fifth, physicians often manage cancer treatment planning processes and therefore are charged with ordering third party services to generate work product for assessing next steps in the process. Here, physicians apply judgement and rely on past experiences applied to new or changing patient conditions to assess next steps and, in many cases, there are no clear dependencies within the overall system so that the physician's decision making points end up slowing down the overall treatment planning process.

Sixth, it is known that cancer state factors (e.g., diagnosed cancer, location of cancer, cancer stage, other cancer characteristics, other user conditions (e.g., age, gender, weight, race, genetics, habits (e.g., smoking, drinking, diet)), other pertinent medical conditions (e.g., high blood pressure, other diseases, etc.), medications, other pertinent medical history, current side effects of cancer treatments and other medications, etc.) and combinations of those factors render some treatments more efficacious for one patient than other treatments or for one patient as opposed to other patients. Awareness of those factors and their effects is extremely important and difficult to master and apply, especially under the pressure of time constraints when delay can appreciably affect treatment efficacy and even treatment options and when there are new insights into treatment efficacy all the time.

Seventh, in many cases complex and time consuming processes are required to identify factors needed to select optimized cancer treatments and initiation of some of those processes is dependent on the results of prior processes. For instance, a tumor sample has to be collected from a patient prior to developing a genetic panel for the tumor, the panel has to be completed prior to analyzing panel results to identify relevant factors and the factors have to be analyzed prior to selecting treatments and/or clinical studies to select for a specific patient.

The complexity of treatment selection processes and advantages associated with expedited selection and treatments have made it impossible for a physician to independently understand, develop and consider all relevant factors in a vacuum and more and more physicians are relying on expert third party service providers to perform diagnostic and data development tests and analysis and identify cancer state treatment options and trial options. To this end, an exemplary service provider may accept orders from physicians to perform genetic tests on patient and tumorous tissues, obtain clinical cancer state data for specific patients, analyze test results along with other cancer state factors, identify optimized treatment and trial options and generate reports usable by the physicians to make optimized decisions. The tasks associated with provider services are diverse, each requiring substantial expertise and/or experience to perform. In many cases tasks required to fulfill a service request include a plethora of both manual and automated tasks performed by different provider entities where many tasks cannot be initiated until one more other tasks are completed (e.g., one task may rely on data and information generated by five other tasks to be initiated). For these reasons, providers typically employ many differently skilled experts and automated systems to perform tasks, one expert or system handing off results to the next to facilitate a sequence of processes.

In many cases these service providers are used by many physicians and the number is growing precipitously as testing and results analysis become more complex and the results more informative and valuable to cancer state diagnosis and treatment prescriptions. The sheer volume of service orders that has resulted has led to cases where providers are having difficulty meeting service request demands in a timely fashion. The press of time has led to development of best service practices whereby a provider follows very specific sequential processes in an attempt to efficiently complete tasks required to intake orders and ultimately generate timely reports. An exemplary order process for developing genetic patient and tumor data, considering that data in conjunction with other cancer state factors, selecting treatment recommendations and/or clinical trial recommendations and reporting to a physician may take 2 or more weeks and may include the following sequenced sub-processes.

First, a physician prepares and faxes a requisition form to a service provider which is manually entered into a spreadsheet pursuant to an order entry process. Here, periodically, excerpts of the spreadsheet are provided to a wet lab process and a report generation process indicating samples which are expected and the processing instructions for those samples. At some later date (e.g., a few days later), the wet lab process receives patient and tumor samples from the physician or from a pathology laboratory which are accessioned into a spreadsheet and notifications of the sample accessions are pushed to an order process, a variant science process, and the report generation process.

A pathology specialist reviews the samples and enters details into the spreadsheet and that data is pushed to the report generation process. Pursuant to the wet lab process, the samples are prepared for sequencing and are put into the sequencer and analysis instructions are pushed to the variant call process. A bioinformatics process waits for sequencer output and analyzes patient data test data and then pushes results and instructions to a variant categorization process. The variant categorization process performs analysis on patient data and pushes data to a clinical therapies process and a clinical trials process as well as to the report generation process. The clinical therapies process curates treatment recommendations which are pushed to the report generation process. In parallel, the clinical trials process curates treatment recommendations which are also pushed to the report generation process. The report generation process, having captured all of the data, produces a final report which is reviewed by a specialist and then pushed out to the order process for delivery to the requesting physician.

While scripted push type sequenced processes like the one described above have some advantages, they also have several shortcomings. First, in general, data push type systems are a problem because each data producer process typically needs to conform to the requirements of at least one and in many cases several consumer processes. This leads to a double-bottom-line struggle for the producer, which, in addition to being concerned with the production of specific data itself, also needs to adapt to constraints of the consumer processes (e.g., is affected by time requirements of the consumer process, has to provide data in a format suitable for the consumer process, etc.). This problem is amplified when a producer process must push data to multiple consumer processes, adapting to the constraints of each.

Second, in a push type system, if data or a push notification is lost, in many cases it is difficult to detect that event (e.g., if a stochastic notification is not received or properly recorded, how can the lack of notification be detected?).

Third, the above exemplary push type order process only describes a perfectly operating sequence where each of the processes produces correct data on a first attempt and where process handoffs between provider entities are seamless. In reality problems routinely occur in complex order processes and sequences. In a push type system, at least some producer processes need to push additional signals to other affected business processes, generally upstream processes which have already executed. This results in a circular dependency where a process A depends on a process B, and process B also depends on process A. Circular dependencies tend to result in excessive coupling between processes. Adding handling of exception flows to a push-centric model tends to result in an overabundance of dependencies, where most processes know about most other processes. This overabundance of dependencies is a burden to allowing any process iteration which is required in many cases and under many sets of circumstances.

Fourth, in known systems, many data pushes consist of manual tasks (e.g., manual handoff steps), such as hand entering data into a spreadsheet, taking excerpts of a spreadsheet and emailing them to a colleague in another business unit, passing printouts between teams, etc. Manual handoff of data occurs generally because the pattern of pushing data between processes requires a large number of complex notifications. In cases where a process iterates, necessary iterations often occurs faster than systems can be built to adapt to the messages, especially when considering exception flows.

Fifth, the exemplary push type system allows for the complete instruction set for a downstream consumer to materialize within a producer process which obscures any understanding of how an order will be or has been processed.

Sixth, in a push type system where processes are built based on decentralized instructions, mismatches between producer processes and consumer processes have been known to inadvertently occur, especially in cases where processes are extremely complex.

Seventh, in push type systems, producers routinely push data forward to consumer processes. Here, in order to handle processing loads efficiently, each process tends to place incoming data onto a queue and, as a result, each process creates and maintains its own data and task queueing mechanism so that the system maintains many redundant queues.

Eighth, processes in a push type system are generally self-contained other than accepting pushes and sending pushes to other external processes. These self-contained processes are generally responsible for tracking their own inputs and outputs, and for capturing and indexing data products appropriately. Ideally, all these push type processes would preserve the most important data including data useable to link through the processes from an originating order to ultimate data products in oncological reports resulting in perfect bookkeeping. In practice, this has not been the case and, in many cases, it has proven difficult to unambiguously join a process's data products with an originating order and final report.

Ninth, the sheer volume of cancer related studies, trials, and new relevant technologies routinely leads to new insights, procedures and processes. Each new insight, procedure or process may need to be worked into an existing process sequence. In a push system reworking a sequence is complex as different consumers have different requirements that need to be supported and therefore, in many cases, new insight, process and procedure support is delayed and patients cannot quickly benefit from those types of developments.

Tenth, while a third party service provider can define and support “optimized reports” for physicians, in many cases there will be a range of acceptable process sequences and report types given circumstances and therefore different physicians or specific institutions may have process and report preferences. In a scripted push type system it is difficult to support many different client process and report preferences.

### SUMMARY OF THE DISCLOSURE

A disclosed adaptive order system includes an order management system, such as a genomic test processing system, that receives basic initial service request information from a physician and uses that information to generate complex and fully defined system orders suitable to drive an entire process associated with patient record intake, genetic sequencing and other tests, variant calling and characterization, treatment and clinical trial selection and reporting. Among other things, an exemplary order includes a set of business processes referred to hereinafter as “items” that must be performed in order to generate data products that are required to either instantiate a completed instance of an oncological report as an end work product or that are needed as intervening data required to drive other order item completion. Embodiments herein are directed to a disease state of cancer. However, other embodiments may capture other disease states, including, for example: diseases or other health conditions, such as cancer, cardiovascular disease, diabetes and other endocrine diseases, skin disease, immune-mediated diseases, stroke, respiratory disease, cirrhosis, high blood pressure, osteoporosis, mental illness, developmental disorders, digestive diseases, viruses, bacterial infections, fungus infections, or urinary and reproductive system infections.

In at least some embodiments, the order management system may include one or more order management engines with order templates which specify specific items for specific order types as well as dependencies (e.g., which items depend on completion of other items to be initiated). For instance, for an exemplary order, the order management system automatically selects either one or several template types required to fulfill an order. For example, an order may require two different DNA tests and each test may correspond to a different template that maps out a sequence of items to be completed. In this case, both test templates would be used to generate an order map that combines items from each template. Where several templates are selected, the management system is programmed to identify duplicate items and where possible, remove duplicate items from an eventual system order.

In particularly advantageous embodiments the adaptive order system, such as the order management engines, may also include an “order hub” that receives and stores orders from the order management system and thereafter manages the entire adaptive order system per order items, dependencies, and other information. The adaptive system has been developed for use with a distributed order processing system including a plurality of microservices where each microservice performs one or more items to yield one or more data products. As several examples, an exemplary accession sample item tracks receipt of a physical specimen from a patient and physician, a variant call item tracks completion of a pipeline that is managed by a bioinformatics team, and a variant characterization item tracks completion of a variant characterization analysis, etc.

The order hub tracks item completion and determines when all dependencies for each item have been successfully completed. Once dependencies have been completed for a specific item, the order hub broadcasts a notification that the specific item can be initiated by one of the microservices that is responsible for completing items of the specific type. A broadcast may be sent directly to a microservice via a direct notification system or generally to all microservices via an indirect notification system. The microservice that performs the specific service either immediately performs the item or adds the item to a queue to be performed once microservice resources required to perform the item are available. One of the microservices initiates the item and, upon initiation, transmits an “in progress” notification to the order hub that the service has been initiated. Where data products from other completed items are required, the microservice accesses those data products. Microservices may be implemented on one or more order processing engines having a receiving and broadcasting engine for receipt and broadcast of any direct notifications and an execution engine for processing the item. For example, the system may include an order management engine and one or more processing engines. The processing engines may include a receiving engine to receive a state of an order from the order management engine, an execution engine to determine a sequence of steps to advance the received state of an order to a final state, to iteratively designate each step of the sequence of steps as completed before initiating a next step of the sequence of steps, and to advance the state of the order to a final state when a last step of the sequence of steps is completed, and a broadcasting engine to broadcast the final state of the order to the order management engine. The order management engine may cause one of the processing engines to generate a next-generation sequencing report from the final state of the order.

The processing engines, in one example, may include a first processing engine that receives the state of an order indicating DNA processing of a specimen and a second order processing engine that receives the state of an order indicating RNA processing of the specimen, where the DNA and/or RNA processing may include collecting a sample (e.g., by scraping a prepared FFPE slide to collect a sample of the sample's tissue, by removing cells from a liquid biopsy specimen to collect a cell-free sample of the specimen, by extracting peripheral whole blood, etc.), isolating DNA and/or RNA nucleotides from the same, amplifying the isolated nucleotides, sequencing the amplified nucleotides, mapping the sequenced nucleotides to a reference genome such as a human reference genome, identifying genetic variants from the reference genome in the sequenced nucleotides and/or measuring an abundance of at least one of the mapped nucleotides, and generating a report from the identified genetic variants and/or from the measure abundance of the mapped nucleotides.

In another example, the processing engines may include a first processing engine that receives a state of an order indicating DNA processing of a normal specimen and a second processing engine that receives a state of an order indicating DNA processing of a tumor specimen. The normal or tumor specimen processing may include collecting a sample, isolating normal or tumor DNA nucleotides from the relevant same, amplifying the isolated nucleotides, sequencing the amplified nucleotides, mapping the sequenced nucleotides to a reference genome such as a human reference genome, identifying genetic variants from the reference genome in the sequenced nucleotides and/or measuring an abundance of at least one of the mapped nucleotides, and generating a report from the identified genetic variants and/or from the measure abundance of the mapped nucleotides.

Upon completion of an item, such as when the item or order has been advanced to a final state for the current specific item, a microservice transmits an item “item complete” notification to the order hub indicating that the item has been completed. In addition, the microservice stores the data product in one or more system database(s) for subsequent access by other items or other system services generally.

In particularly advantageous systems the order hub only performs a limited set of tasks including storing and monitoring orders and order item statuses and generating notifications to system microservices in order to initiate item processing when dependencies are met. Thus, in some systems the order hub never receives data products and microservices simply store generated data products in a network access storage (NAS) system (e.g., Amazon Web Services (AWS) cloud based Simple Storage Service (S3)).

In some cases the notification that an item is complete and its data product(s) is stored in a database takes the form of a fulfillment address that indicates the virtual network location of the data product. Here, the order hub uses the fulfillment address as an item status indication and, in at least some embodiments, when a microservice executing another item requires the data product, the microservice polls the order hub for the fulfillment ID (e.g., the address at which the data product has been stored), receives the fulfillment ID, and then uses that ID to access the required data product. In other cases where microservices and the order hub use identical database address formats for data storage and retrieval, when a microservice requires a data product generated by another item, the microservice will have enough information from the order hub notification and other sources to resolve the database address or location at which the data product is stored without requiring additional information from the order hub.

In at least some embodiments the order hub maintains an audit log that tracks orders and item activities. For instance, each time a new order is created or an existing order is modified (e.g., items are added to or deleted from the order), a distinct and time stamped audit record may be generated memorializing the order change. Similarly, any order item status change event such as when an order is initiated (e.g., in progress), completed, cancelled, paused, or deemed low quality (e.g., a quality control (QC) fail) for any reason, a distinct and time stamped audit record may be generated and stored to memorialize the order status event change.

In at least some cases order hub may use the audit log to generate a visual representation of a current status of an order and/or a time based historical visual representation of order status. For instance, in some cases a directed acyclic graph (DAG) representation may be generated that includes a set of item icons or DAG vertices representing order items where the vertices are linked together by process flow lines or edges to indicate when one item is dependent on others. In some cases item vertices will be distinguished with short item labels and may be color coded or otherwise visually distinguished based on item status at a time associated with a specific view of the order status. For instance, if a system user selects a view of a first order on Mar. 13, 2019 which corresponds to a time when the first order was partially completed, the DAG representation may use different colors to highlight item icons indicating not initiated, in progress, complete, QC fail and pause statuses. Other visual representations are contemplated.

In one aspect, the present disclosure may relate to a method for conducting genomic sequencing that includes the step of storing a set of user application programs wherein each of the programs requires an application specific subset of data to perform application processes and generate user output. The method also may include, for each of a plurality of patients that have cancerous cells and that receive cancer treatment, the steps of: (a) obtaining clinical records data in original forms where the clinical records data includes cancer state information, treatment types and treatment efficacy information, (b) storing the clinical records data in a semi-structured first database, (c) for each patient, using a next generation genomic sequencer to generate genomic sequencing data for the patient's cancerous cells and normal cells, (d) storing the sequencing data in the first database, (e) shaping at least a subset of the first database data to generate system structured data including clinical record data and sequencing data wherein the system structured data is optimized for searching, (f) storing the system structured data in a second database, (g) for each user application program: (i) selecting the application specific subset of data from the second database; and (ii) storing the application specific subset of data in a structure optimized for application program interfacing in a third database.

The method also may include the step of storing a plurality of micro-service programs where each micro-service program includes a data consume definition, a data product to generate definition and a data shaping process that converts consumed data to a data product, the step of shaping including running a sequence of micro-service programs on data in the first database to retrieve data, shape the retrieved data into data products and publish the data products back to the second database as structured data. In addition, the method may include storing a new data alert in an alert list in response to a new clinical record or a new micro-service data product being stored in the second database. Each micro-service program may monitor the alert list and determine if stored data is to be consumed by that micro-service program independent of all other micro-service programs, and at least a subset of the micro-service programs may operate sequentially to condition data. At least a subset of the micro-service programs specify the same data to consume definition. Additionally, the step of shaping may include at least one manual step to be performed by a system user, where the system adds a data shaping activity to a user's work queue in response to at least one of the alerts being added to the alert list. At least one of the micro-services may be a variant annotation service.

The first database may include both unstructured original clinical data records and semi-structured data generated by the micro-service programs. Additionally, each micro-service program may operate automatically and independently when data that meets the data to consume definition is stored to the first database.

The application programs may include operational programs, and at least a subset of the operational programs may include a physician suite of programs usable to consider cancer state treatment options. At least a subset of the operational programs may include a suite of data shaping programs usable by a system user to shape data stored in the first database, and the data shaping programs may be for use by a radiologist and/or a pathologist. The method may make use of a set of visualization tools and associated interfaces usable by a system user to analyze the second database data.

The third database may include a subset of the second database data, and the third database may include data derived from the second database data.

The method also may include the steps of presenting a user interface to a system user that includes data that indicates how genomic sequencing data affects different treatment efficacies.

Each cancer state may include a plurality of factors, and the method may further include the step of using a processor to automatically perform the step of analyzing patient genomic sequencing data that is associated with patients having at least a common subset of cancer state factors to identify treatments of genomically similar patients that experience treatment efficacies above a threshold level. Additionally or alternatively, the method further including the steps of using a processor to automatically identify, for specific cancer types, highly efficacious cancer treatments and, for each highly efficacious cancer treatment, identify at least one genomic sequencing data subset that is different for patients that experienced treatment efficacy above a first threshold level when compared to patients that experienced treatment efficacy below a second threshold level.

The application programs may include operational programs. At least one of the operational programs may be a variant annotation program. At least one of the operational programs may be a clinical data structuring application for converting unstructured raw clinical medical records into structured records.

The data vault database may include a database of molecular sequencing data. The molecular sequencing data may include DNA data, RNA data, normalized RNA data, tumor-normal sequencing data, variant calls, variants of unknown significance, germ line variants, MSI information, and/or TMB information.

The method further may include determining an MSI value for the cancerous cells, determining a TMB value for the cancerous cells, identifying a TMB value greater than 9 mutations/Mb, detecting a genomic alteration that results in a chimeric protein product, detecting a genomic alteration that drives EML4-ALK, determining neoantigen load, identifying a cytolytic index, distinguishing a population of immune cells (dependent: TMG-high/TMB-low), determining CD274 expression, and/or reporting an overexpression of MYC.

The method also may include detecting a fusion event, which may be a TMPRSS-ERG fusion.

The method may include the step of detecting a PD-L1 in a lung cancer patient.

The method may include indicating a PARP inhibitor, which may be for BRCA1 or for BRCA2.

The method may include the step of recommending an immunotherapy. The recommended immunotherapy may be one of CAR-T therapy, antibody therapy, cytokine therapy, adoptive t-cell therapy, anti-CD47 therapy, anti-GD2 therapy, immune checkpoint inhibitor and neoantigen therapy.

The cancer cells may be from a tumor tissue and the non-cancer cells may be blood cells. Alternatively, the cancer cells may be cell free DNA from blood. The cancer cells may be from fresh tissue, from a FFPE slide, from frozen tissue, or from biopsied tissue.

In another aspect, a method for conducting genomic sequencing may include the steps of, for each of a plurality of patients that have cancerous cells and that receive cancer treatment: (a) obtaining clinical records data in original forms where the clinical records data includes cancer state information, treatment types and treatment efficacy information; (b) storing the clinical records data in a semi-structured first database; (c) obtaining a tumor specimen from the patient; (d) growing the tumor specimen into a plurality of tissue organoids; (e) treating each tissue organoids with an organoid specific treatment; (f) collecting and storing organoid treatment efficacy information in the first database; (g) using a processor to examine the first database data including organoid treatment efficacy and clinical record data to identify at least one optimal treatment for a specific cancer patient. The method also may include the steps of storing a set of user application programs wherein each of the programs requires an application specific subset of data to perform application processes and generate user output, shaping at least a subset of the first database data to generate system structured data including clinical record data and organoid treatment efficacy data wherein the system structured data is optimized for searching, storing the system structured data in a second database, for each user application program, selecting the application specific subset of data from at least one of the first and second databases and storing the application specific subset of data in a structure optimized for application program interfacing in a third database. Further, the method may include the steps of using a genomic sequencer to generate genomic sequencing data for each of the patients and the patient's cancerous cells and storing the sequencing data in the first database, where the step of examining the first database data includes examining each of the organoid treatment efficacy data, the genomic sequencing data and the clinical record data to identify at least one optimal treatment for a specific cancer patient.

In either aspect, the sequencing data may include DNA sequencing data and/or RNA sequencing data. In either aspect, the sequencing data may include only DNA sequencing data or only RNA sequencing data. Sequencing may be conducted using the xT gene panel or using a plurality of genes from the xT gene panel. Sequencing alternatively may be conducted using at least one gene from the xF gene panel, using the xE gene panel, or using at least one gene from the xE gene panel.

Sequencing may be done on the KRAS gene, the PIK3CA gene, the CDKN2A gene, the PTEN gene, the ARID1A gene, the APC gene, the ERBB2 gene, the EGFR gene, the IDH1 gene, the CDKN2B gene, or the TP53 gene. Similarly, sequencing may be performed on a particular cancer type.

Sequencing may include MAP kinase cascade, EGFR, BRA, or NRAS.

### BRIEF DESCRIPTION OF THE SEVERAL VIEWS OF THE DRAWINGS

FIG. 1 is a schematic illustrating a genomic order processing system that is consistent with at least some aspects of the present disclosure;

FIG. 2 is a schematic illustrating an exemplary order map and system sub-processes that is consistent with at least some aspects of the present disclosure;

FIG. 3 is similar to FIG. 2, albeit showing a more complex order map that include additional order items;

FIG. 4 is a schematic illustrating a DNA NGS tumor/normal template item sequence that is used to instantiate new item based orders that is consistent with at least some aspects of the present disclosure;

FIG. 5 is similar to FIG. 4, albeit showing a DNA tumor only exemplary whole exome NGS panel template;

FIG. 6 is similar to FIG. 4, albeit showing a DNA tumor only preview exemplary solid tumor NGS panel template;

FIG. 7 is similar to FIG. 4, albeit showing a DNA liquid biopsy exemplary liquid biopsy NGS panel template;

FIG. 8 is similar to FIG. 4, albeit showing an RNA tumor only template;

FIG. 9 is similar to FIG. 4, albeit showing an immunohistochemistry (IHC) mismatch repair (MMR) template;

FIG. 10 is a schematic illustrating exemplary order, order-item, item and item dependency format specifications that are consistent with at least some embodiments of the present disclosure;

FIG. 11 includes a flowchart that shows an order instantiation process performed by the intake system shown in FIG. 1;

FIG. 12 is a flowchart illustrating an order management process that is performed by the order hub server shown in FIG. 1;

FIG. 13 is a flowchart illustrating an item processing process that is performed by one of the microservices that is shown in FIG. 1;

FIG. 14 is a schematic that illustrates the FIG. 2 variant calling process in more detail;

FIG. 15 is a schematic illustrating an audit record format specification that is consistent with at least some aspects of the present disclosure;

FIG. 16 is a schematic illustrating a user interface screen shot and a visualization tool that enables a user to view a current or historical order map and order item statuses;

FIG. 17 is similar to FIG. 16, albeit showing the order map at a later point in time;

FIG. 18 is similar to FIG. 17, albeit showing the order map at a later point in time;

FIG. 19 is similar to FIG. 18, albeit showing the order map at a later point in time;

FIG. 20 is similar to FIG. 19, albeit showing the order map at a later point in time; and

FIG. 21 is similar to FIG. 20, albeit showing the order map at a later point in time.

### DETAILED DESCRIPTION OF THE DISCLOSURE

The various aspects of the subject disclosure are now described with reference to the drawings, wherein like reference numerals correspond to similar elements throughout the several views. It should be understood, however, that the drawings and detailed description hereafter relating thereto are not intended to limit the claimed subject matter to the particular form disclosed. Rather, the intention is to cover all modifications, equivalents, and alternatives falling within the spirit and scope of the claimed subject matter.

In the following detailed description, reference is made to the accompanying drawings which form a part hereof, and in which is shown by way of illustration, specific embodiments in which the disclosure may be practiced. These embodiments are described in sufficient detail to enable those of ordinary skill in the art to practice the disclosure. It should be understood, however, that the detailed description and specific examples, while indicating examples of embodiments of the disclosure, are given by way of illustration only and not by way of limitation. From this disclosure, various substitutions, modifications, additions rearrangements, or combinations thereof within the scope of the disclosure may be made and will become apparent to those of ordinary skill in the art.

In accordance with common practice, the various features illustrated in the drawings may not be drawn to scale. The illustrations presented herein are not meant to be actual views of any particular method, device, or system, but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or method. In addition, like reference numerals may be used to denote like features throughout the specification and figures.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof. Some drawings may illustrate signals as a single signal for clarity of presentation and description. It will be understood by a person of ordinary skill in the art that the signal may represent a bus of signals, wherein the bus may have a variety of bit widths and the disclosure may be implemented on any number of data signals including a single data signal.

The various illustrative logical blocks, modules, circuits, and algorithms acts described in connection with embodiments disclosed herein may be implemented as electronic hardware, computer software, or combinations of both. To clearly illustrate this interchangeability of hardware and software, various illustrative components, blocks, modules, circuits, and acts are described generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the embodiments of the disclosure described herein.

In addition, it is noted that the embodiments may be described in terms of a process that is depicted as a flowchart, a flow diagram, a structure diagram, or a block diagram. Although a flowchart may describe operational acts as a sequential process, many of these acts can be performed in another sequence, in parallel, or substantially concurrently. In addition, the order of the acts may be re-arranged. A process may correspond to a method, a function, a procedure, a subroutine, a subprogram, etc. Furthermore, the methods disclosed herein may be implemented in hardware, software, or both. If implemented in software, the functions may be stored or transmitted as one or more instructions or code on a computer-readable medium or media. Computer-readable media includes both computer storage media and communication media including any medium that facilitates transfer of a computer program from one place to another.

It should be understood that any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed there or that the first element must precede the second element in some manner. Also, unless stated otherwise a set of elements may comprise one or more elements.

As used herein, the terms “component,” “system”, “engine”, and the like are intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a computer and the computer can be a component. One or more components may reside within a process and/or thread of execution and a component may be localized on one computer and/or distributed between two or more computers or processors.

The word “exemplary” is used herein to mean serving as an example, instance, or illustration. Any aspect or design described herein as “exemplary” is not necessarily to be construed as preferred or advantageous over other aspects or designs.

Furthermore, the disclosed subject matter may be implemented as a system, method, apparatus, or article of manufacture using standard programming and/or engineering techniques to produce software, firmware, hardware, or any combination thereof to control a computer or processor based device to implement aspects detailed herein. The term “article of manufacture” (or alternatively, “computer program product”) as used herein is intended to encompass a computer program accessible from any computer-readable device, carrier, or media. For example, computer readable media can include but are not limited to magnetic storage devices (e.g., hard disk, floppy disk, magnetic strips . . . ), optical disks (e.g., compact disk (CD), digital versatile disk (DVD) . . . ), smart cards, and flash memory devices (e.g., card, stick). Additionally it should be appreciated that a carrier wave can be employed to carry computer-readable electronic data such as those used in transmitting and receiving electronic mail or in accessing a network such as the Internet or a local area network (LAN). Of course, those skilled in the art will recognize many modifications may be made to this configuration without departing from the scope or spirit of the claimed subject matter.

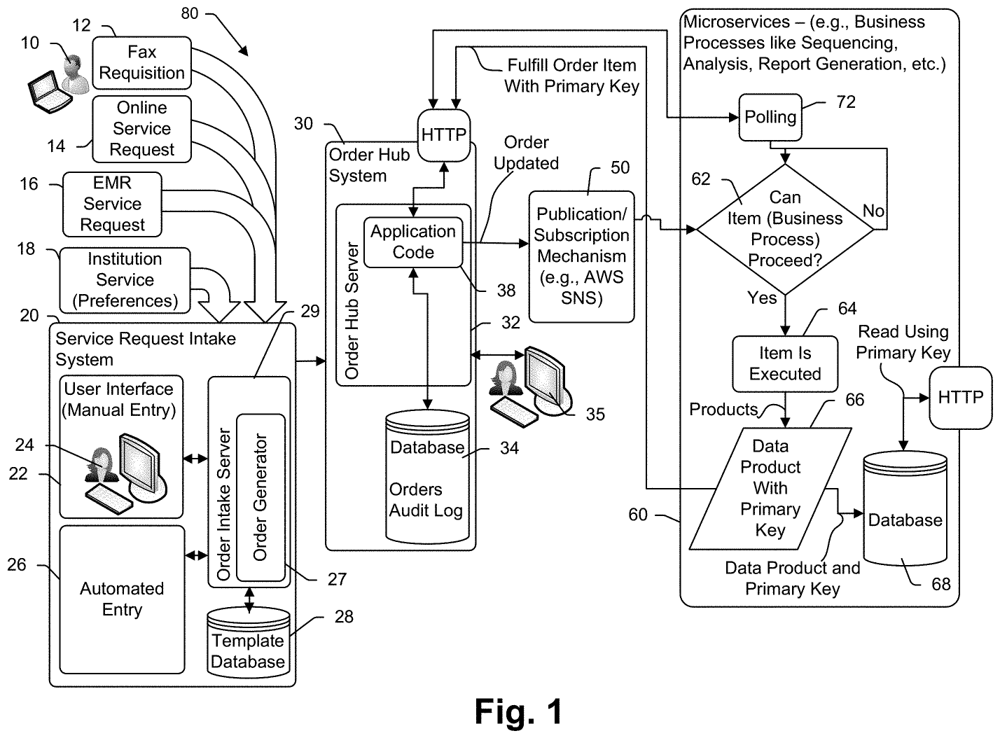

Referring now to the drawings wherein like reference numerals correspond to similar elements throughout the several views and, more specifically, referring to FIG. 1, the present disclosure will be described in the context of an exemplary adaptive order processing system 80 that is consistent with at least some aspects of the present disclosure. Processing system 80 includes several subsystems or functional components including a service request intake system 20, an “order hub” 30, a publication/subscription mechanism 50 and a plurality of microservices collectively identified by numeral 60. The intake system 20 receives service requests from physicians and converts those requests to system orders that specify system processes required to generate data products needed to fulfill the requests, ultimately generating one or more final reports that are delivered to the requesting physician. The service orders are provided to order hub 30 and specifically to an order hub server 32 which stores the orders in a database 34 and runs application code 38 to manage tasks associated with each order by notifying microservices 60 when tasks are to be performed and tracking task execution and completion. Order hub 30 also facilitates an archiving or audit log function whereby order changes (e.g., modifications including a new order and order changes as well as order item status changes including pending, in progress, complete, failed, paused and cancelled) are tracked and stored where a system user can access the audit log information to assess current status of order tasks as well as to see a current or historical (e.g., at a specific point in time) visual representation of order item statuses and an order item map.

Referring again to FIG. 1, request intake system 20 includes an order intake server 29, a template database 28 and sub-systems for receiving and manipulating received service requests including an automated entry sub-system 26 and an abstractor specialist interface 22. Service requests can be received in several different ways including, for instance, a fax requisition system 12, an online service request system 14, or an EMR service request system 16. Other ways of acquiring service requests are contemplated.

In some cases requested services will require system 80 to acquire or access detailed patient clinical or medical history records and in these cases a service request will include the required record data or some way to access or to obtain that data. For instance, in some cases a service request will include a copy of a patient's clinical records or a link to a records server that can be used to access those records.

System 80 requires data and information in a defined or normalized format. In some cases service requests and clinical records are received in the normalized format required by system 80 and in those cases the requests and clinical records are consumed by server 29 as received. For instance, in the case of a service request received from an EMR system, the EMR system may be programmed to generate clinical data in the normalized format required by system 80. In other cases, service requests and clinical records may not be in the normalized format but may be in a format that can be automatically converted to the normalized format via automated entry subsystem 26. In other cases clinical data may be generally unstructured or in a format that cannot be automatically converted to the normalized format and in that case an “abstractor” specialist (e.g., service provider employee charged with converting requests and clinical records to the normalized formats) manually converts a received order and records to the system required format. For instance, where an unstructured service request is received via facsimile, an abstractor specialist may glean request information therefrom and enter order information in via interface 22 in the normalized format for consumption by the system 80. In still other cases automated entry system 26 may be capable of converting at least some request and record information into the normalized formats and an abstractor specialist may be charged with confirming accuracy of that information as well as filling in any information that cannot be automatically converted. Abstractor software programs/interfaces have been specially designed to facilitate abstraction.

A typical service request will identify a specific set of tests or other procedures to be performed by the service provider. In at least some cases specific physicians or institutions (e.g., medical facilities at which physicians work) have preferences for test types, test sub-processes, report types, report formats, etc. Referring still to FIG. 1, in addition to receiving service requests, intake system 20 has access to institution preferences 18 which may specify any of specific test types, sub-processes, report types and formats. In FIG. 1 the phrase “institution preferences” is used generally to refer to specific physician preferences as well as general institutional preferences. In at least some cases it is envisioned that there will be a hierarchy of preferences where a specific physician's preferences may take precedence over institutional preferences or vice versa. In a case where no preferences exist, the system will implement a set of default preferences for any received order or may have a feedback mechanism whereby any required preference is sought from an ordering physician or institution.

Referring again to FIG. 1, intake system 20 converts a service request and institution preferences into a system service order (hereinafter “order”) that includes a plurality of business processes or discrete tasks that can be completed to generate data or information needed to prepare one or more final reports. Hereinafter, unless indicated otherwise, a system business process will be referred to as an “item” and therefore an order may be represented as a series of consecutive items and parallel items. Hereinafter, when an item is complete, the item is said to be “fulfilled” and, in most cases that means that the item has generated one or more data products that have been stored in a system database for subsequent access by other items that comprise a common service order.

In exemplary embodiments, service request intake system 20, order hub system 30, publication/subscription mechanism 50, microservices 60, and corresponding elements of FIG. 1 may reside in a physical laboratory at a geographic location such as a country, state, county, city, or building or may reside in a cloud based architecture without a designated geographic location such as AWS, Microsoft Azure, Google Cloud, Alibaba Cloud, Oracle Cloud, IBM Cloud, or other cloud-based architectures. In another exemplary embodiment, some of service request intake system 20, order hub system 30, publication/subscription mechanism 50, microservices 60, and other corresponding elements of FIG. 1 may reside at one or more geographic locations while others reside in one or more cloud based architectures without departing from the spirit of the disclosure herein.

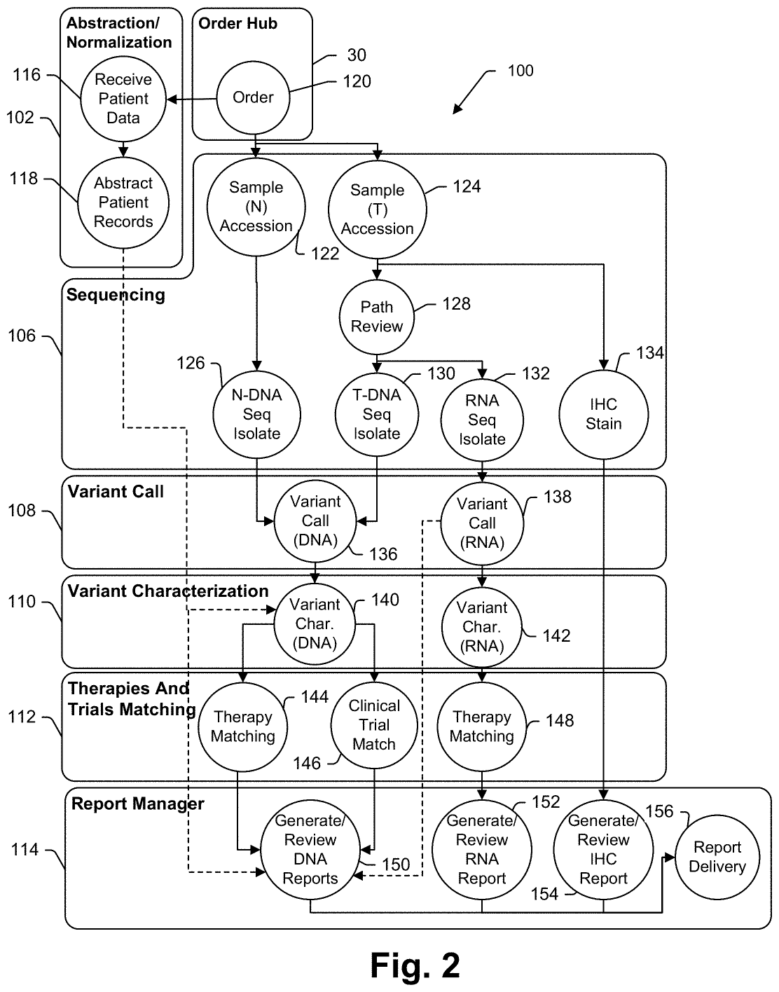

Referring now to FIG. 2, an exemplary and simplified order map 100 is illustrated that includes items related to an exemplary set of genetic tests and report generation tasks. The ordered map 100 is related to a specific order 120 generated by intake system 20 and stored in order hub database 34 (FIG. 1) and includes items that are grouped into item subsets that together define order sub-processes geared toward partial completion of the service order. To this end, see that the order items in FIG. 2 are grouped together by sub-processes including an abstraction/normalization sub-process 102, a sequencing sub-process 106, a variant calling sub-process 108, a variant characterization sub-process 110, a therapies and trials matching sub-process 112 and a report management sub-process 114.

The arrows between items in FIG. 2 indicate order flow and item dependencies where any item immediately downstream of any other item can only be initiated once the prior item(s) has been completed (e.g., downstream items are “dependent”). In at least some cases hereafter the relationship between any item and an immediate downstream item will be referred to as a parent-child relationship where immediately adjacent upstream and downstream items are parent and child items, respectively. All child items are “dependent” on their directly linked parent items and child items “depend” on or from their parent items while parent items are “dependencies” of their directly linked child items.

Referring again to FIGS. 1 and 2, order hub 30 tracks progress and completion of each item in each system order, determines when all dependencies for each item are complete and, when all dependencies for an item are complete, publishes an “item ready” notification on a system network shared with microservices 60 indicating that the item is ready to be initiated.

Each microservice 60 includes resources that are capable of completing at least one and in many cases several different types of order items. For example, a sequencing lab may comprise a microservice where the lab is capable of generating many different sequence panels for many different sample types including human normal, human tumor, human organoid, human stool, human saliva, human blood, human buffy coat, human CHIP, spinal cord fluid, other human fluid, etc., which may be analyzed in fresh or processed form. In these cases the lab may be capable of performing one or more items of different types for sample and panel pairs. Examples of processed specimens include but are not limited to FFPE slides, extracted DNA, and extracted RNA. As another example, a bioinformatics lab that performs variant call items may be capable of performing many different item types depending on sequencing lab tests, institutional preferences, etc. As another example, the order items may support various types of testing or analysis, such as surveillance testing, screening testing, and diagnostic testing.

As another example, the order items may support various types of testing or analysis, including but not limited to comprehensive genomic profiling, hot-spot panel testing, early stage breast cancer testing, hereditary breast and ovarian cancer (“HBOC”) testing, whole genome sequencing, low-pass whole genome sequencing, low-pass whole genome sequencing with DNA methylation, liquid biopsy, PCR testing, IHC staining, etc.

Each microservice may be fully automated or may include automated and manual resources. For instance, in many cases automated systems generate intra-item data products that a pathology or other system specialist need to consider, confirm and in some cases modify, as part of the item process. In at least some cases on microservice may use other microservices as resources to perform various tasks.

In some embodiments a single service provider provides all microservice resources and handles all order items. In other cases, one service provider may provide order hub 30 and a subset or none of the microservice resources while other service providers provide other microservices required by the system. Thus, for instance, a first service provider may manage order hub 30 at a first location while NGS sequencing items may be performed by microservices at a hospital or pathology lab at a second location, bioinformatics processing items may be performed by microservices operated by a bioinformatics service provider and/or automated bioinformatics method(s) operating at a third location, and variant calling and characterization items may be performed by microservices operated by a variant science service provider and/or variant method(s) operating at a fourth location. Other permutations are also possible (e.g. NGS sequencing at one location and everything else at a second location). In each case each location uses the same order hub information to assist in the automatic processing of information in order to complete testing processes. For instance if sequencing and bioinformatics are conducted at different locations, order hub 30 “listens” on the network for sequencing items to be complete before triggering child bioinformatics items.

Referring still to FIG. 1, microservices 60 subscribe to the order hub publications and listen on the network for “item ready” notifications and, when an item is ready to be initiated, if a specific microservice is capable of executing the item, initiates the item and sends an “in progress” status notification to order hub 30. Order hub 30 retransmits the “in progress” notification to other system microservices so that other microservices similarly capable of executing the item stand down to avoid item duplication. Thus, the exemplary FIG. 2 order map 100 can be thought of as a set of tasks to perform or, from the perspective of order hub 30, a map of order items to be tracked.

Referring again to FIG. 2, abstraction and normalization sub-process 102 includes items for receiving patient data 116 (e.g., tracking receipt of clinical or medical record data for a patient) and abstracting 118 and normalizing that data (e.g., tracks completion of extracting details about a patient from clinical documents) to generate data in a normalized or structured, system-useable format. The abstracted data is stored in a system database and is rendered accessible to other downstream system items via the internet or other communication network.

Exemplary sequencing sub-process 106 includes seven items and commences with tumor and normal sample accession items 124 and 122, respectively. Normal sample accession item 122 tracks receipt of a patient's normal physical specimen from a physician or biorepository. In some cases the sample may be a tissue sample and in other cases the sample may be a substance refined from a tissue or fluid specimen, the tissue, fluid specimen, or refined substance may be referred to as an “isolate”. In another embodiment, a fluid sample, such as a liquid biopsy may be cultivated from a specimen such as by a blood draw. A liquid biopsy may be processed in a centrifuge to separate cells from non-cells in the liquid biopsy, the non-cell remains may be siphoned off of the cell material after centrifuging. In other words, the non-cellular portion may be obtained by removing any cells from the liquid biopsy specimen to collect a cell-free sample of the specimen. In some instances, a cell-free sample of a specimen may merely be substantially cell-free and trace amounts of cells may remain. Nucleotides may be isolated and amplified in accordance to standard DNA or RNA procedures. This process includes receiving and accessioning a sample into the lab and includes categorization (e.g., tumor or normal) and source (human/mouse) of the expected sample as well as collection of case specific sample information (e.g., Institution ID, patient info, case #, sample block #, sample ID, order information (Tumor/Normal, DNA, RNA, Immuno tests, etc.). Tissue sample accession item 124 tracks receipt of a patient's tumorous physical specimen from a physician.

Path review item 128 tracks completion of a pathological review of a tumor sample (e.g., tissue or isolate) where the review entails diagnosis of the accessioned tumor sample and is fulfilled with storage of a diagnosis record. More specifically, a hemotoxylin and eosin (H&E) slide deck is collected, slides are verified to ensure that what is reported on the pathology report is what is shown in the slides. A pathology specialist updates diagnosis (e.g., refines from cancer or breast cancer to Invasive ductal carcinoma) and adds tumor cell counts and tumor purity metrics to the data set. The pathology specialist maps the pathology report data and the added information to an internal structured data format for internal records.

The sequencing items track sequencing of a specific sample. Each sequencing item causes a lab to load a sequencer with samples scraped from slides which have been RNA/DNA separated and amplified and with controls (controls testing for contamination, biases, etc., for quality control). Controls are tested to conform with required accuracy for identifying a corresponding variant call in the control sample to ensure successful sequencing in each batch.

Regarding the specific sequencing items, the N-DNA Seq Isolate item 126 tracks that sequencing of a patient's normal physical sample is imminent. Here, an isolate is prepared from a patient's normal sample for a specific panel (e.g., exemplary whole exome NGS panel; exemplary solid tumor NGS panel; exemplary liquid biopsy NGS panel, etc.) and DNA combination and for a specific coverage depth (e.g., high/low) and is placed in a flow cell destined for a service provider's genomic sequencers. This item is fulfilled with microsevice storage of a sequencer isolate record and raw sequencer output files. Similarly, T-DNA Seq Isolate item 130 tracks that sequencing of a patient's tumorous physical sample is imminent. In this case, an isolate is prepared from a patient's tumorous sample for a specific panel and DNA combination and is placed in a flow cell destined for a service provider's genomic sequencers. RNA Seq Isolate item 132 tracks that sequencing of an isolate from a patient for a specific panel and RNA combination is imminent and that an isolate is likewise placed in a flow cell for sequencing. IHC stain item 134 tracks completion of a staining of slides for an IHC report, scanning and uploading the slide and pathological review of the slide.

Referring again to FIG. 2, variant call sub-process 108 includes two items including a variant call DNA item 136 and a variant call RNA item 138. Item 136 tracks completion of a DNA pipeline that is managed by a bioinformatics team and, as known in the industry, is completed using the sequencer outputs related to the Isolate items 126 and 130. This item analyzes the upstream sequence isolate fulfillments and sequencer output files and is fulfilled (e.g., is completed so that a data product is stored and available for use by other order items) by an analysis which describes any mutations in a patient's DNA and the RNA variant call is fulfilled in a similar fashion. Item 138 tracks completion of an RNA pipeline and is completed using the sequencer output related to isolate items 132.

Variant characterization sub-process 110 includes two items including a variant characterization DNA item 140 and a variant characterization RNA item 142. Item 140 tracks completion of a DNA variant characterization analysis which analyzes the upstream variant call fulfillment and is fulfilled with an analysis which describes the pathology of the mutations in a patient's DNA. Item 142 tracks completion of an RNA variant characterization. RNA variant characterization is completed using the variant calls produced by item 138. Several characterization processes are associated with items 140 and 142. Exemplary characterization processes include S/M(NP) processes to identify single and multiple nucleotide polymorphisms (e.g., variations), an InDels process to detect insertions in and deletions from the genome, a CNV process to detect and identify one or more copy number variations, a fusions process to detect gene fusions, a TMB process to calculate a tumor mutational burden score, an MSI process to calculate a microsatellite instability score, and an IHC process. Once variants are characterized, each variant is then classified by the characterization item as benign, likely benign, pathogenic, likely pathogenic or VUS (e.g., variant of unknown significance). Each item 140 and 142 stores characterized variants along with the classifications at which point the variant classification process is complete.

Therapies and Trials Matching sub-process 112 includes three items in the FIG. 2 map including a DNA related therapy matching item 144, a clinical trial matching item 146 and an RNA related therapy matching item 148. The therapy matching items 144 and 148 track completion of DNA and RNA based therapy recommendations in which detected variants are matched with therapies that specifically treat those variants. Trials matching item 146 analyzes upstream variant categorization fulfillment and is itself fulfilled with storage of recommendations of clinical trials that may benefit a patient. Trial matching involves matching detected variants to clinical trials that have inclusion criteria for the specific variants.

Report manager sub-process 114 includes items that, in general, generate a final report, check quality of the report, facilitates a sign-out process for the report and delivers the report to an ordering physician. More specifically, the report manager sub-process brings together the results from each order “branch” (e.g., RNA, IHC, DNA, etc.) and causes references from one branch to the other where appropriate to generate data needed to develop a final report. Report manager sub-process 114 then facilitates data error checking to ensure that all needed report branches exist and have passed quality control. The manager sub-process creates an unpopulated shell report based on order objectives, test types performed, etc.

An artificial intelligence program auto-populates the shell based on rulesets and information curated via machine learning. Sub-process 114 enables a pathology specialist to confirm or modify AI populated report information and add additional information derived during review. Once done reviewing and supplementing the report, the pathology specialist signs and time stamps the report and then a PDF of the report is generated, structured data from the report is made available in an online portal display, etc. In some cases sequencing reports for variant calls and characterizations are made independently available in both machine and human readable forms. In some cases treatment reports are generated as clinical trial reports. Sub-process 114 may also facilitate report deliveries via e-mail, posting of alerts, etc.

In FIG. 2, report manager sub-process 114 includes four items. A first item 150 is a generate/review DNA reports item which tracks consumption of upstream variant categorization, match clinical trials, match therapies, path review and sequence isolates fulfillments, generation of a report and sign-off by a pathology specialist and is fulfilled with a final report. For example, item 150 within sub-process 114 may include accessing a database of information of individuals with a health condition similar to that of the patient who was the source of the specimen from which the DNA report was generated in order to include at least some of that information in the final report. Similarly, items 152 and 154 track completion and sign-off of an RNA sequencing report and an IHC report as well as generation of PDF reports and uploading of those reports to an attachments service. Once a report PDF has been generated, that PDF must be delivered to an order management team and the report delivery item 156 tracks status of that delivery. Report delivery item 156 is fulfilled with a delivery confirmation ID.

Referring still to FIG. 2, the microservice executing each item completes or fulfills the item by generating a database storage location or “address” at which to store item data product(s), storing the data product(s) at the generated address and then transmitting the address as a fulfillment ID to order hub 30. Here, in at least some cases the address is based on a system wide address format so that other items that have access to information that populates the address can independently resolve the address without requiring information from the order hub. For this reason, “item ready” notifications can be extremely simple and only need to identify limited information useable to distinguish one order item from other items queued at the hub for execution.

Some examples may utilize the disclosure herein to establish a distributed order management system. For example, a physician may place a test order through her electronic medical record interface. The test order originates from the electronic medical record through EMR service request 16 and is stored in order hub system 30. The order hub system creates an item associated with the specimen that ultimately will be analyzed as part of the test order. The item tracks the processing of the specimen where it is stored in a biorepository, such as a pathology lab, and continues to track the specimen as it is either analyzed at the biorepository lab (e.g. with the processes tracked by 106) or is shipped to a testing lab for analysis. In the case where the specimen is analyzed at the biorepository, the order hub system 30 may be integrated or otherwise operatively coupled to the biorepository's management system, such as its LIMS system. An item created by the order hub system may track completion of the analysis (e.g. completion of sequencing) and provide for the results of the analysis (e.g. sequencing files such as BAM files) to be transferred to another location for further analysis and processing (e.g. variant calling 108). In some cases, the processing done subsequent to specimen analysis (such as variant calling or variant characterization) may be performed by different institutions or companies, and so it should be understood that the order hub system may be additionally integrated or otherwise operatively coupled to the management systems of such companies in order to track those activities as they are conducted at such institutions or companies. The various processes may continue until report delivery, which may be returned to the ordering physician's electronic medical record in a format known in the art (such as an HL7 format).

In some examples, the processing of a specimen may be dependent upon the patient herself. For example, with testing that involves the patient providing a specimen (e.g. a saliva or stool specimen), the order hub system 30 may be integrated into software available to the patient through, for instance, the patient's smart phone. An item may be generated by the order hub system 30 to track the shipment status of the specimen collection kit (such as by tracking messaging notifications provided by the shipping company), such as whether it was successfully delivered to the patient's home or whether, once the specimen was acquired and placed in the collection kit, the specimen was successfully picked up from the patient's home. An item may be generated by the order hub system 30 to track whether the patient had successfully acquired a specimen (which may be tracked, for example, by providing a graphical user interface such as a button within an app on the patient's smart phone, whereby the patient presses the button to indicate that the specimen was successfully acquired).

In some examples, the processing of a specimen may be dependent on a third party. For example, with testing that involves the collection of a blood sample, a mobile phlebotomy laboratory technician may visit the patient's house to acquire a blood sample. The technician may have access to software through her smart phone that is integrated or otherwise operatively coupled to the order hub system 30 such that the order hub system 30 can generate one or more items to track the interaction between the technician and the patient (and reflect the circumstances giving rise to the status of the order, such as whether the patient was home or not for the visit; whether the patient complied or refused to comply with the blood draw; whether blood was successfully acquired; whether the blood specimen was shipped; and so forth).

Although FIG. 2 and its accompanying steps 106, 108, 110, 112, and 114 are provided to give a detailed description of an order map, it should be understood that the order map provided in FIG. 2 illustrates a specific type of order and that other orders may have order maps that differ in the nature of the processing required in order to achieve a final report or result.

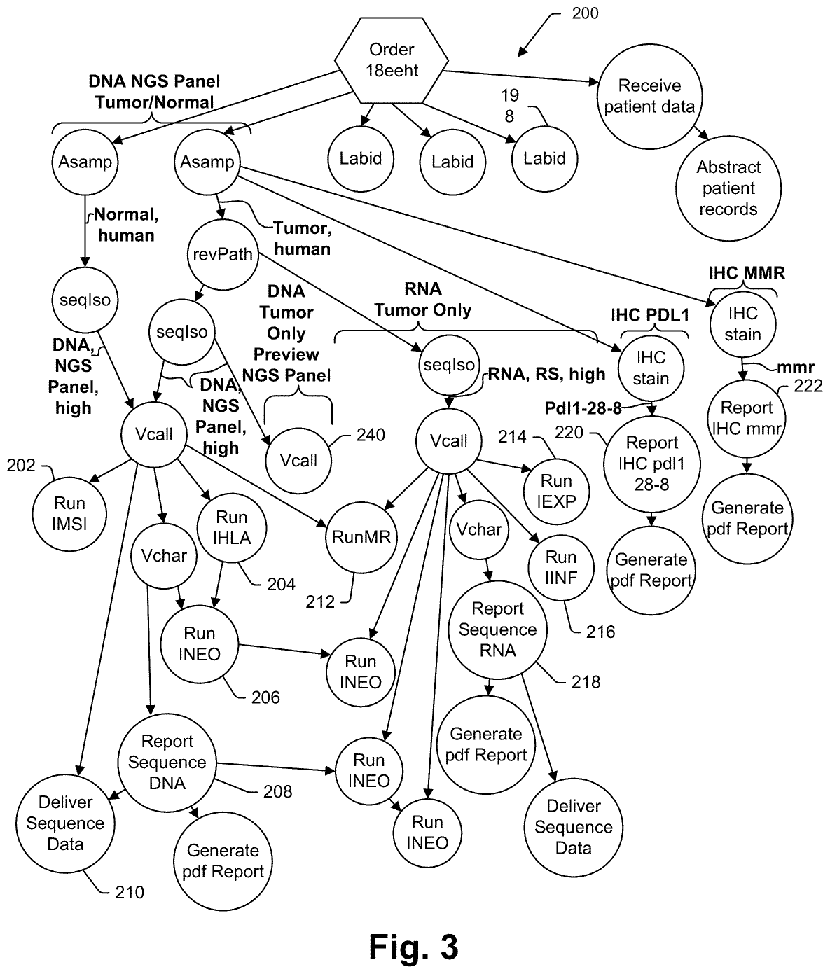

Referring now to FIG. 3, a more complex service order 200 is illustrated that again includes a circular representation for each item in the order. In FIG. 3, some of the item labels are identical to the FIG. 2 item labels and refer to the same items. Because the illustrated order 200 includes many more items than shown in FIG. 2, several abbreviated item labels are used in FIG. 3 so that the entire order map can be shown. To be clear, label “Asamp” in FIG. 3 corresponds to “Sample Accession” in FIG. 2, “seqIso” in FIG. 3 corresponds to “Seq Isolate” in FIG. 2, “revPath” in FIG. 3 corresponds to “Path Review” in FIG. 2, “Vcall” in FIG. 3 corresponds to “Variant Call” in FIG. 2, “Vchar” in FIG. 3 corresponds to “Variant Char” in FIG. 2, and “Generate pdf Report” in FIG. 3 corresponds to “Generate/Review Reports” in FIG. 2.

FIG. 3 includes other common order items including a “lab identification” item 198 that tracks a lab identification process to identify the lab from which a sample is received as well as any special steps that need to be taken with respect to the identified lab which could affect the order map, a “Run IMSI” item 202 (e.g., tracks execution of an Immunotherapy MSI analysis module), a “Run IHLA” item 204 (e.g., tracks execution of an Immunotherapy HLA analysis module), several “Run INEO” item 206 (e.g., each tracks execution of an Immuno-neoantigen analysis module), a “Report Sequence DNA” item 208 (tracks completion and sign-out of a DNA sequencing report), several “Deliver Sequence Data” items 210 (e.g., each tracks delivery of raw sequencing data results to a client), a “Run MR” item 212 (e.g., tracks execution of an MR analysis module), a Run IEXP item 214 (e.g., tracks execution of an Immunotherapy Expression Targets analysis module), a “Run IINF” item 216 (e.g., tracks execution of an Immunotherapy Infiltration), a “Report Sequence RNA” item 218 (tracks completion and sign-out of an RNA sequencing report), a “Report IHC pdl1 28-8” item 220 (tracks completion and sign-out of an Immunohistochemistry Report using PDL1 28-8 stain) and a “report IHC mmr” item 222 (tracks completion and sign-out of an IHC report for mismatch repair detection. While the map in FIG. 3 includes several order item types, many more item types are contemplated, some of which are described hereafter in more detail. In addition, while order 200 is somewhat complex, far more complex orders including hundreds of items are contemplated.

Referring again to FIG. 1, to simplify the process of converting a service request into a system order including a mapped set of items, item mapping templates have been developed that encapsulate item sequences that commonly appear within order maps. To this end, most service requests specify sequencing tests to be performed where the test set is selected from a small set. The tasks or items associated with each of the tests are typically duplicated each time the test is performed and therefore, templates have been developed for a small set of archetypes of tests. For example, in a particularly advantageous system there are four main archetypes of tests and each can be represented by an archetype specific item sequence. The four archetypal tests include DNA solid tumor sequencing (each of a whole exome NGS panel, a solid tumor NGS panel and another exemplary NGS panel), DNA liquid biopsy sequencing (liquid biopsy NGS panel), RNA sequencing and an IHC test.

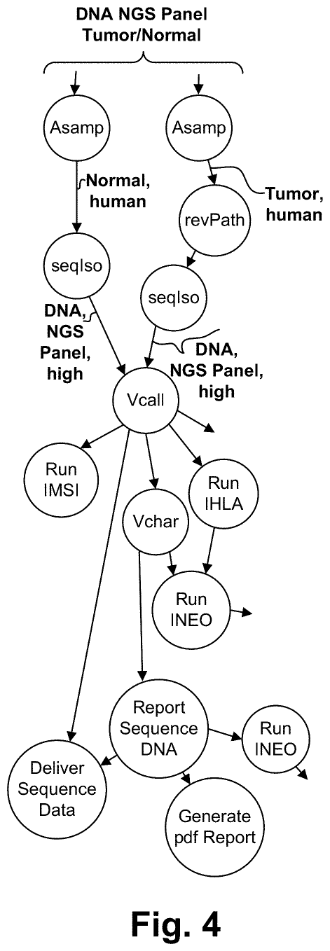

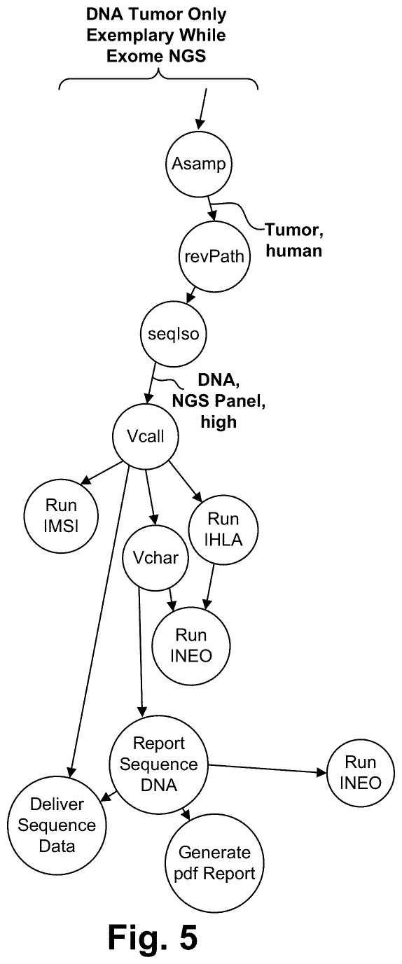

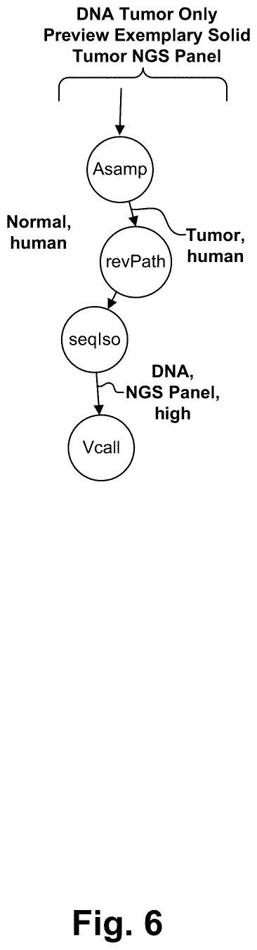

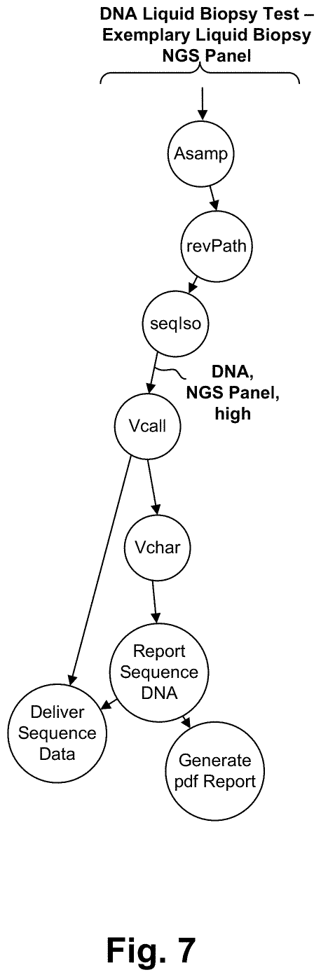

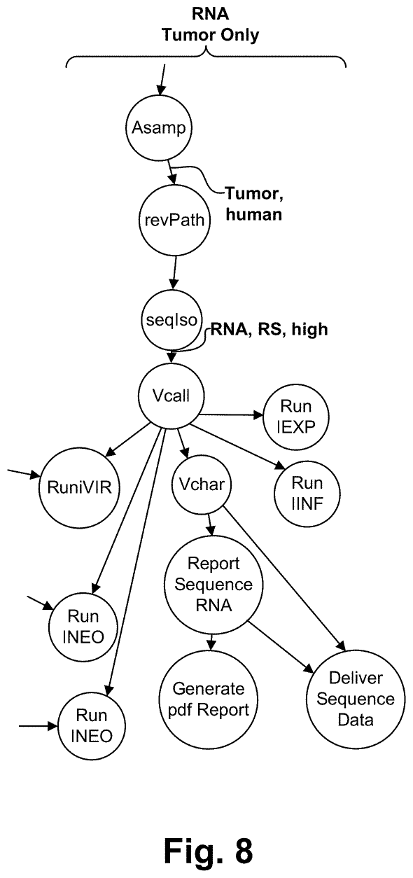

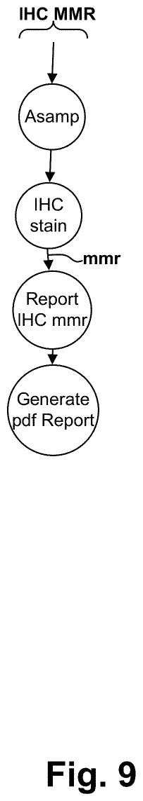

Referring now to FIG. 4, an exemplary item sequence template that corresponds to a DNA NGS tumor/normal match panel is illustrated which includes normal and tumor sample accession items, a pathology review item, two sequence isolate items, a variant call item, a variant characterization item Run IMSI, IHLA and INEO items and a set of reporting items. FIG. 5 shows an exemplary item sequence template that corresponds to a DNA tumor only whole exome NGS panel and FIG. 6 shows an exemplary item sequence template that corresponds to a DNA tumor only preview NGS panel. FIG. 7 shows an exemplary DNA liquid biopsy NGS panel. FIG. 8 shows an RNA test item sequence and FIG. 9 shows an exemplary IHC test item sequence.

Comparing the template item sequence from FIG. 4 to the left portion of FIG. 3 (see portion labelled DNA NGS Panel Tumor/Normal) it should be appreciated that the entire FIG. 4 sequence is included in FIG. 3. The replication of FIG. 4 in FIG. 3 is because the FIG. 4 template was used along with other test templates to generate the multi-test FIG. 3 order map. In fact, each of the FIG. 4, FIG. 6, FIG. 8 and FIG. 9 templates was used by an operating service 20 request intake system to generate the FIG. 3 order map. Comparing the FIG. 6 template item sequence to map 200 in FIG. 3 it should be appreciated that only a portion of the template sequence persists in map 200. To this end see that the only FIG. 6 template item that is reflected in map 200 is a “Vcall” item 240 which is labelled “DNA tumor Only Preview NGS panel”. Similarly, several of the items in the FIGS. 7 and 8 templates are missing in map 200. FIGS. 6, 8 and 9 template items are missing in map 200 because they were duplicative with other items that occur as part of the DNA NGS Panel Tumor/Normal item mapping from the FIG. 4 template. Thus, upon receiving a service request, order intake server 27 identifies requested tests, accesses an item template for each archetypal test, then identifies duplicative items among test templates and links child items together through a single duplicative parent item whenever possible in order to eliminate duplicative system processes and tasks in the final order map.

Referring again to FIG. 3, another test type referred to as an IHC PDL1 is included in exemplary order map 200 which is not associated with any one of the exemplary archetypal templates in FIGS. 4 through 8. Here, the IHC PDL1 test item sequence may be defined by institutional preferences or may be specified by an abstractor specialist or other system administrator based on service request requirements.

In at least some embodiments other factors in addition to requested tests and institutional or physician preferences are used by intake system 22 to generate an order map. For instance, in at least some cases the intake system will discern whether or not clinical data for a patient associated with a received service request exists within the system databases and will add items to an order map required to create a new patient and abstract required data when that data does not exist within the system databases. In addition, information on a requisition form may be used to add items to an order, to delete items from a template item sequence or to modify default template items. Moreover, in at least some cases billing details for an institution or physician may be obtained and used to modify order items.

In many cases institutional preferences indicate if an order is for research or clinical use which influences whether report items like “report sequence DNA”, “report sequence RNA”, “report IHC-mmr”, etc., and related items are added to an order map. Institutional preferences also may specify tests that an ordering physician can add to a service request which limits possible template item sequences that can be added to an order map. Institutional preferences also specify if raw sequencing data should be delivered to an institution which determines if a “deliver sequence data” item will be added to a map for an institution and parameters for that item. Institutional preferences also may specify if RNA and DNA tumor samples will be received separately which influences whether RNA and DNA tests will share the same “accession sample” item or if a separate accession sample item will be created for each test. Other institution preferences may be considered and appropriately handled by the order hub system.

Ordering physician preferences may specify contact preferences and a care team which affects what is reported out to a client, the manner of report (e.g., e-mail, facsimile, other) and to whom reports are sent. Other physician preferences may specify default tests typically ordered for patients (e.g., NGS panel matching+MMR+PDL1). Another preference may indicate if raw sequencing data should be delivered to an ordering physician which determines if a “deliver sequence data” item is added for the physician as well as parameters for that item.

While only four archetypal test templates are described above, it is contemplated that a system may include tens if not hundreds of item sequence templates for different purposes or functions. For instance, another simple exemplary item sequence template may be provided to help manage patient record ingestion and abstraction processes. Here, an exemplary sequence of template items may include “Receive patent data” (e.g., receipt of clinical patient documents/records), “Abstract patient” (e.g., abstract patient timeline) and “Quality review patient” (e.g., patient record is reviewed by a manager, may result in further actions being created for the patient). As another instance, an item sequence template may be provided to collect and optionally backfill (e.g., full or partial re-run of bioinformatics or variant science) data for a specific patient test that is part of a pharma deliverable. Here, an exemplary template item sequence may include “Identify asset and test”, “Capture variant call”, “Capture variant characterization” and “Collect Pharma Data”.

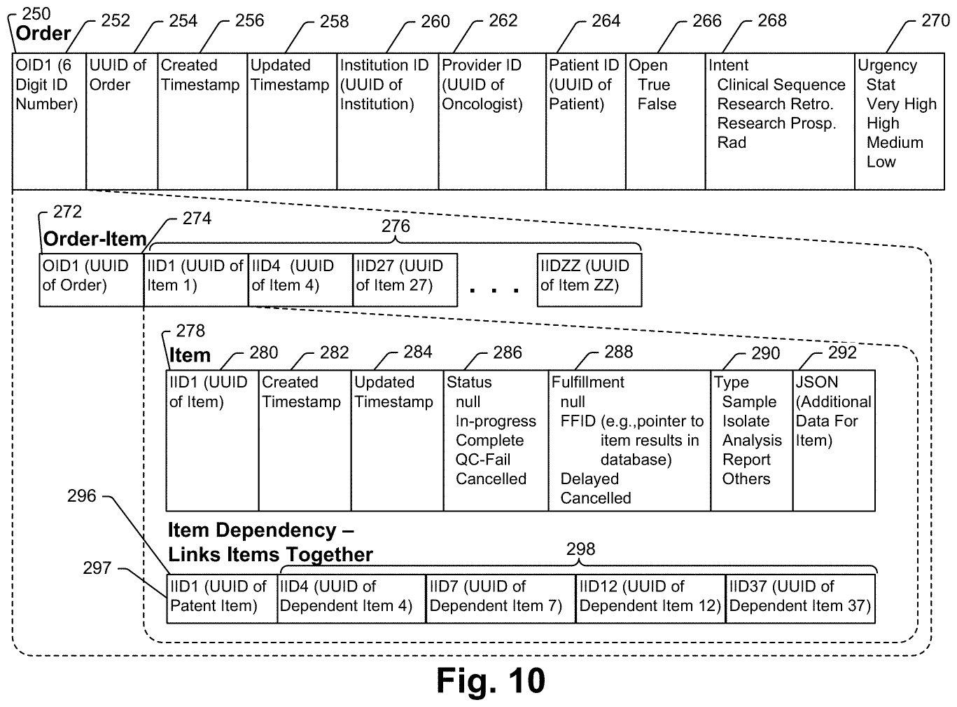

Referring again to FIG. 1, order hub 30 stores received orders in database 34. Referring also to FIG. 10, in the disclosed system an exemplary system order includes a set of related data constructs including an order specification 250 (hereafter an “order”), an order-item specification 272 (hereinafter an “order-item list” or simply “item list”), an item specification 278 (hereafter an “item”) and an item dependencies specification 296 (hereafter a “dependencies list”). Exemplary order 250 includes a data format including ten information fields 252 through 270. Each system order is assigned a human-readable unique identifier which, in the disclosed system, is a 6 character value like “18eeft” (see again the order at the top of FIG. 3) and that unique identifier is placed in the first order field 250 for uniquely identifying the order. A second field 252 is populated with an assigned 4 character universal unique identifier (UUID) which is used as an internal system key and which is guaranteed by the system to be immutable.

Referring again to FIG. 10, a third field 256 includes a “created timestamp” that indicates the time at which an order was initially created and fourth field 258 includes an “updated timestamp” indicating the most recent update to an order or execution of any item associated with the order. Fifth field 260 includes an institution ID which is also a 4 character UUID that uniquely identifies the institution with which a requesting physician is associated, sixth field 262 includes a provider ID which includes a 4 character UUID that uniquely identifies the service provider that manages the order processing system 80, and seventh field 284 includes a 4 character patient UUID identifying the patent associated with the order. Eighth field 266 includes an order “open” status indicating whether or not an order is completed where possible field values include “True” (e.g. that the order is not complete) and “False” (e.g., that the order is not open and therefore has been completed). Ninth field 268 includes an “intent” field in which a value indicates an intended use of an order. Intent values may be any one of clinical sequence, research retrospective, research prospective and radiation. The intent is not used for order prioritization in at least some system embodiments. Tenth field 270 is an “urgency” field and is used to indicate a desired order processing speed and is used to prioritize system orders. Exemplary urgency field 270 values arranged from highest to lowest include stat, very-high, high, medium, low and very-low. Order hub 30 uses a triage process to control order and order item sequences among all pending and in progress orders based on the urgency values.

As described above each system order includes an item map (see exemplary map 200 in FIG. 3) that includes a plurality of items. A separate order-item list 272 is provided for each order 250 and includes a list of items to be executed to complete the order. The list 272 includes a specific order UUID in a first field 274 and a separate item in each of a second through N fields collectively labelled 276. Each item in one of the second through N fields is identified by its unit UUID.

Each item is defined by a separate item specification. In FIG. 10 one exemplary item specification is shown at 278 for the first item in the order-item list 272. Exemplary item specification 278 includes seven fields. An item's UUID is placed in the first item specification field 280 and created and updated timestamps are placed in second and third fields 282 and 284, respectively. The fourth field 286 operates as an item status field where one value selected from a list including null, in-progress, complete, delayed, QC-fail and cancelled is placed in the field to specify a current item status. In-progress, complete, delayed (e.g., that the item is taking longer to complete than expected or is waiting for something to occur to continue execution), and cancelled status values should be understood. A null value indicates that an item has not been initiated (e.g., either item dependencies have not been completed or no microservice has indicates an in-progress status). A QC-fail status is related to quality control and indicates that the system or a system administrator has determined that an item has failed for some reason. In the case of a failed item, the system may automatically attempt to complete the failed item again once or several times or may perform some type of administrator notice process so that an administrator can address the failure.

Referring still to FIG. 10, fifth item specification field 288 is a “fulfillment” field which indicates if a task has been completed and may have any of null, cancelled, or a fulfillment ID, values. “Null” means an item has yet to be fulfilled and “cancelled” means the item has been cancelled for some reason. A fulfillment ID indicates that the item has been completed. The fulfillment ID includes a database pointer address (e.g., specifies a database location) indicating a location at which data products associated with completion of an item have been stored.

Sixth field 290 is a “type” field indicating a type of an associated item. The type defines many aspects about an order item, such as additional fields that may be populated within an item specification, types of item fulfillment(s) allowed, and a number and type of items that may be present in the dependencies list (see 297 in FIG. 10). JSON field 292 is an additional item data field that is optional in at least some embodiments.

Item dependencies list 297 defines item dependencies for an associated item (e.g., parent items/tasks within a common order that need to be completed prior to commencement of another item). In list 297, an item identified in a first list field 296 is dependent on completion of all of the items in the second through N fields collectively labelled 298. Thus, in FIG. 10, there is a separate order-item specification 272 for each order specification 250, a separate item specification 278 for each item listed in specification 272 and also a separate item dependency specification 297 for each item listed in specification 272.

Table 1 in Appendix A presents a list of item types and related information. Some of the listed item types are referenced above in the context of the order maps in FIGS. 2 and 3 and others are described in Table 1 for the first time. For each item type the Table 1 information indicates how the item needs to be fulfilled, required dependencies (e.g., a minimum set of other items that need to be completed to execute a specific item type), an exemplary item type data format and additional information required to instantiate an item of the specific type. For example, for the third item type (3) accession sample in Table 1, the item is only completed once a fulfillment ID that points to a database record for a sample is placed within the item fulfillment field 288 in an associated item specification 278 (see again FIG. 10), the accession sample item type has no dependencies but requires additional information to define the item including tissue classification, tissue source, slide count and slide stain information. While Table 1 includes many different item types, the list is not intended to be exhaustive and the disclosed system is extendable to support other kinds of work. For instance, other items may track any process that includes multi-team item coordination.

It should be appreciated that in the disclosed system, a system order contains only data that is necessary to indicate precisely what items need to be completed to complete the order, the status of those items, and a way to reference ultimate item data products. Completion status and an output reference for each item are encapsulated in a fulfillment ID placed in an item fulfillment field 288. Thus, an order and associated order items do not include details about item data products which intentionally limits the scope of knowledge that order hub 30 has about outside systems. The only knowledge contained in order hub about item data product is the fulfillment ID that points to the data product in a database.

Referring again to FIG. 1, and now more specifically to the microservices 60, each microservice that has subscribed to publication mechanism 50 “listens” for notifications indicating that an item that can be executed by the specific microservice is ready to be executed. Here, again, an item is ready to be executed when all other items from which the specific item depends have been completed. At decision block 62 a microservice determines if a received notification indicates a ready item that the microservice can execute. Where no notification indicates a ready item that the microservice can execute, control simply continually loops through block 62. If a notification indicates a ready item that at least one microservice can execute, at some point a microservice that can execute the item initiates execution 64 of that item. Upon initiating execution of an item, the executing microservice transmits an “in progress” notice back to order hub 30 which then notifies the other system microservice that the ready item is in progress by another microservice to avoid a case where one item is inadvertently duplicated by other microservices. Once an item is completed, a data product and primary key (e.g., database address) for that data product 66 are stored in database 68 for subsequent access by other order items via an HTTP link or the like. In addition, once an item is completed, the microservice transmits the item primary key (e.g., a fulfillment ID in the form of the database address of the associated data product) is transmitted to order hub 30 where it is used to populate the item fulfillment field 28 shown in FIG. 10. Here, the primary key may be transmitted via email, a messenger service, SMS, MMS, broadcast to a bus/network, etc. In at least some cases a microservice is also able to poll 72 order hub data to check item statuses and access other information needed for various purposes.

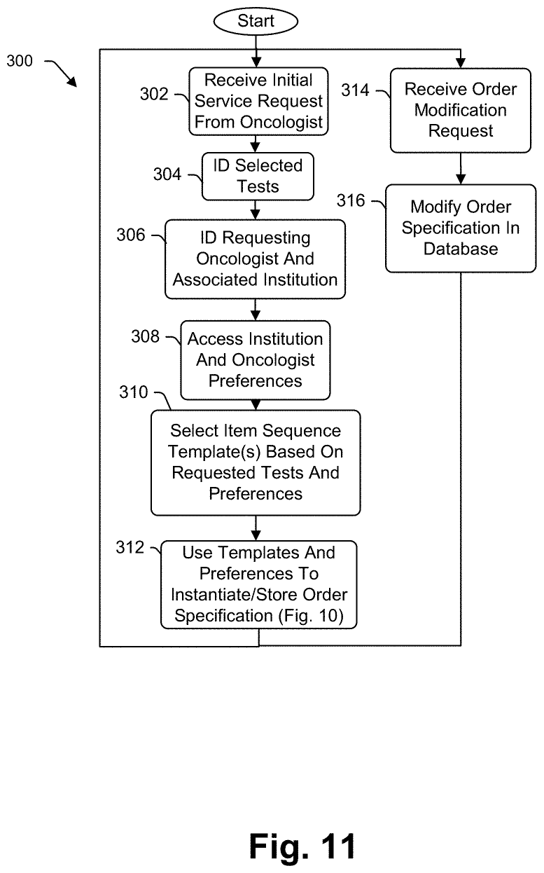

Referring now to FIG. 11 an exemplary process 300 performed by service request intake system 20 (see also again FIG. 1) that is consistent with at least some aspects of the present disclosure for generating a service order is illustrated. At block 302 a physician 10 sends and intake system 20 receives a service request. At block 304 intake server 29 identifies requested tests. At block 306 server 29 identifies the requesting physician and associated institution and at block 308 server 29 accesses institutional and physician preferences. At process block 310 server 29 selects one or more item sequence templates from database 28 based on the requested test, institutional preferences and physician preferences. Here, templates may also in part be based on other information in a requisition form that more specifically define request limitations. At block 312 the templates, preferences and other requisition limitations are used to instantiate and then store an order specification (see again FIG. 10) in order hub database 34 after which control passes back to block 302 where the intake system waits to receive another service request.

Referring still to FIG. 11, in at least some embodiments intake system 20 performs a parallel process including blocks 314 and 316 to receive and consume order changes requested by a physician or a system administrator (e.g., an abstractor specialist). To this end, at block 314 a modification to an existing order is received by the intake system 20. Here, the modification may include cancelling one or more order items, modifying an existing item, adding one or more order items, eliminating an order test or tests, adding a new order test or tests, etc.

At block 316, intake system 20 changes an existing order based on a service request modification. Here, if an order has not commenced prior to reception of a service request modification, the intake system may simple generate a completely new order specification (see FIG. 10) by working through process steps 302 to 312 and then swap the new order specification for the original order specification.

In a case where an original order has been initiated and at least some order items have been completed, intake system 20 may be programmed to assess if any of the completed items and associated data products can be used to fulfill similar items in a modified order. For instance, in at least some cases when a service request modification is received, the intake server may be programmed to execute many of the process steps including blocks 302 through 312 in FIG. 11 anew to generate a completely modified order specification and associated order map. The intake server may then compare the modified order specification with the original order specification to identify identical or common order items and different order items. For order items that appear in the original order specification but not in the modified order specification, the intake server may simply change statuses of those items to “cancelled”. In some cases where one of these cancelled items was previously completed, any fulfillment ID in an item fulfillment field may remain to memorialize that the item was completed and so that the data product associated with that item can be subsequently accessed by other order items.

For order items that appear in the original order specification and are also replicated in the modified order specification, the intake server 29 would leave those item specifications unchanged. Thus, for instance, for an item common between the original and modified order specifications that has already been completed by a microservice, that item would remain fulfilled with a fulfillment ID in the modified order specification. Once an order specification is modified, hub server 32 again commences item management to pick up where execution of the old order left off and to fulfill each of the modified order items.

In other cases when an order amendment is received, intake server 29 may access an original order map (see FIG. 3) and simply amend that map by adding complete item sequences or separate items to the map or by deleting existing item sequences or separate items from the map. In some cases order map amendments may have to be scrutinized by a system administrator prior to reinitiating execution while in other cases execution may be initiated immediately after an amended order map is stored in the order database.

In still other cases a system administrator using an intake system user interface 22 (e.g., a web based computer interface) may be able to access any system order stored in database 34 and modify that order by adding or deleting order items, selecting or cancelling different order tests or other processes, analysis or procedures, changing task parameters, etc. In these cases order changes would be handled in a fashion similar to that described above where a physician makes a service request modification.

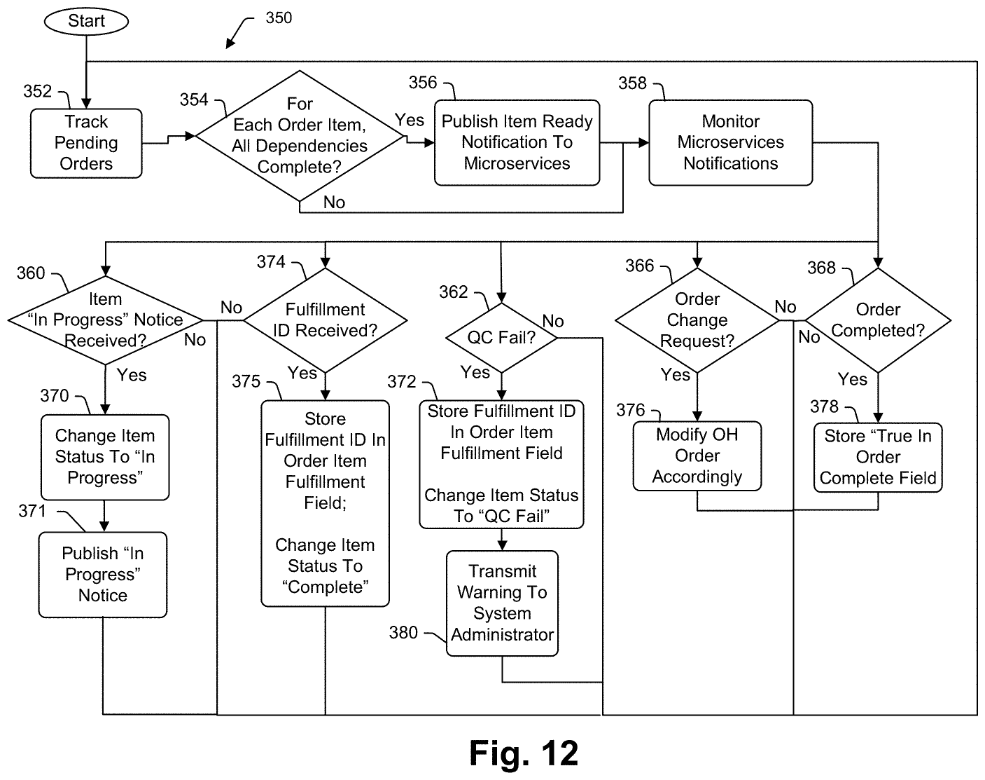

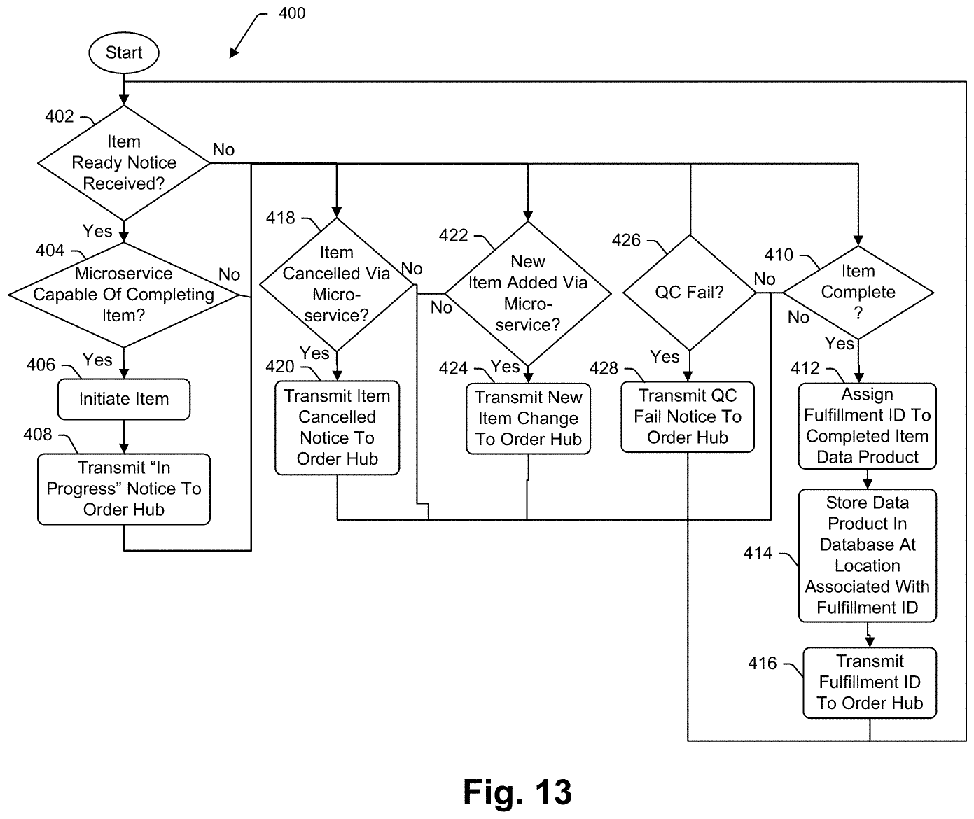

Referring now to FIG. 12, an order hub process 350 for tracking and managing order items performed by microservices is illustrated. FIG. 13 shows a microservice process 400 that operates in parallel at each system microservice 60. FIGS. 12 and 13 will be described together. In FIG. 12, at block 352 the order hub server 32 tracks all pending system service orders and more specifically, all items in all service orders, to assess the status of each item in each order. At block 354, for each item in each order, server 32 determines if all items from which an item depends have been fulfilled (e.g., are complete). If all items from which a specific item depends are complete, control passes to process block 356 where server 32 publishes an “item ready” notification on the network including system microservices 60 after which control passes to block 358 where server 32 monitors for microservice notifications (e.g., notices of item status changes). Referring again to decision block 354, if all items from which a specific item depends are not complete, control passes from block 354 to block 358.

Referring now to FIG. 13 which shows a microservice process 400, at decision block 402 a microservice determines if an “item ready” notification has been received from order hub 30. If an item ready notification has not been received, control passes to other decision blocks as illustrated that are described hereafter. If an “item ready” notification is received, at decision block 404 the microservice determines if the microservice is capable of completing the ready item. If the microservice cannot complete the ready item the microservice simply ignores the “item ready” notification and control passes on to other decision blocks as illustrated. If the microservice can complete the ready item, control passes to block 406 where the microservice initiates the item. At block 408 the microservice transmits an “in progress” notice to order hub 30.

Referring again to FIG. 12, one notification type that may be received at block 358 is an “in progress” notice from a microservice that recently initiated an item. At decision block 360, when an “in progress” notice is received, order hub server 32 control moves to block 370 where the server 32 changes the item status in field 286 (FIG. 10) to “in-progress”. At block 371 server 32 publishes an “in progress” notification to the microservices indicating that one microservice has started the in progress item and therefore that no other microservice should start a duplicative item. After block 371 control loops back up to block 352 where the process described above continues to cycle.

Referring again to FIG. 13, at block 410 the microservice determines if an in process item executed by the microservice has been completed and if so, control passes to block 412. At block 412, the microservice assigns a fulfillment ID to a completed item data product which indicates a system database location/address at which the data product is stored at block 414. At block 416 the fulfillment ID is transmitted to order hub 30.

Referring back to FIG. 12, at decision block 374, order hub server 32 determines if a fulfillment ID has been received from any of the microservices indicating that an item has been completed. Where a fulfillment ID has been received control passes to block 375 where server 32 stores the fulfillment ID in the fulfillment field 288 (see again FIG. 10) and changes the item status in field 286 to complete. After block 371 control loops back up to block 352 where the process described above persists.

In at least some cases some aspect of an item will fail during execution so that an item fails to generate expected data products or so that data products associated therewith are not reliable. In some cases, a pathology specialist or other service provider specialist that performs at least some steps associated with an item may recognize item failure and simply indicate failure via a computer or other type of user input device associated with a microservice. In other cases a system server may be programmed to automatically recognize item failure and flag that failure within the system. For instance, where a data product generated by an item is outside a possible value range, a system processor may recognize the errant value and automatically indicate a QC fail.

In some cases when an item failure occurs, the item and associated order may simply be delayed in a queue until a system administrator can access the item and associated order and address the failure in some fashion such as, for instance, initiating duplicated item. In other cases when an item failure occurs, a microservice may be programmed to automatically initiate a new item of the same type in a second attempt to successfully complete the item. In some cases if item failure persists after a second attempt, a third attempt may occur and so on until a threshold maximum number of attempts result in failure at which point an administrator could be notified.

Where an item fails, a microservice may transmit a “QC fail” notice to order hub 30 so that the hub can memorialize the failure. Similarly, when a duplicative item is initiated, the microservice may transmit a change request to order hub 30 so that hub 30 can memorialize the change. In at least some cases order changes are memorialized within an order. For instance, in the case of an added item, referring again to FIG. 10, the new item may be added to the order-item specification 272 and an item specification akin to specification 278 and an item dependency list akin to 296 may be generated for the new item. As another instance, where an item fails (e.g., a QC fail notice is generated), order hub 30 may place a QC fail status value in the item status field 286 (see FIG. 10) and, where the item generated a data product, may store the data product in a system database and store a fulfilled ID in the item field 288 to memorialize the data result.

In at least some cases a microservice (e.g., automatically or a person affiliated with a microservice) will cancel an order item for some reason. Where an item is cancelled, the microservice may transmit a “cancelled” notice to hub 30 and the hub may memorialize the cancellation. Referring again to FIG. 10, a cancellation is memorialized by changing the status of an item to “cancelled” in field 286 and also by placing a “cancelled” designation in the fulfillment field 288.

Referring again to FIG. 13, an exemplary microservice monitors in progress items at the microservice for any QC fail conditions at block 426 and if a fail condition occurs, control passes to block 428 where the microservice transmits a QC fail notice to order hub 30. Similarly the microservice monitors in progress items at the microservice for any newly added item at block 422 and transmits a new item notice to order hub 30 whenever a new item occurs at block 424 and monitors in progress items at the microservice for any cancelled item at block 418 and transmits a cancelled item notice to hub 30 at 420 whenever any item is cancelled. After blocks 420, 424 and 428 control passes back up to block 402 where the process described above continues to cycle.

Exemplary errors for some of the main order sub-processes shown in FIG. 2 are instructive. To this end, an exemplary sequencing error may involve running a wrong assay through a sequencer, using a wrong sample, a tumor without normal or a normal without tumor situation, etc. In variant calling a common error may be that a data dependency does not exist even if a system generating the dependency indicates that an item has been fulfilled. Similarly, a common variant characterization error may be that a data dependency does not exist even if a system generating the dependency indicates that an item has been fulfilled. A quality control error may occur when a variant calling process detects an error that a sample sequence that was expected is not available. Here, a microservice or provider specialist may request an item to rerun for that sample sequencing and in this case, the system would revise the order map to add additional items into the map to track the modified order items.

In cases where the system or a provider specialist identify errors in a report, those errors may trigger a QC error message to order hub 30 again causing items to be added to a flow for correcting the errors.

In some cases a template error may occur such as, for instance, where tumor and normal branches of an order should have been created but an error caused only the tumor branch to be instantiated in the order map. Here, a normal item sequence would have to be added to the order map. In some cases an order branch has to be completely cancelled from an order map.

Referring again to FIG. 12, QC fails notifications are tracked at block 366 and when one is received hub 30 memorializes the item failure in the item specification (see again item specification 278 in FIG. 10) by storing any fulfillment ID in field 288 for any data product that was generated by the microservice as well as by placing a QC fail indicator in field 286 as indicate data block 377. In addition, in at least some cases a warning signal may be transmitted to an administrator as indicated at block 380 indicating the item that failed.

At block 366 hub server 32 monitors for any order changes (e.g., cancelled items, newly added items, modified items, etc.) and, when an order change is received from one of the microservices, server 32 uses the change information in the notice to modify the order at block 376 by either changing item status to “cancelled”, by adding new items to an order specification, or by amending item characteristics if appropriate.

Referring yet again to FIG. 12, at decision block 368, hub server 32 determines if all items associated with an order have been completed and if so, stores a “True” value in the order specification open field 266 (see FIG. 10 again).

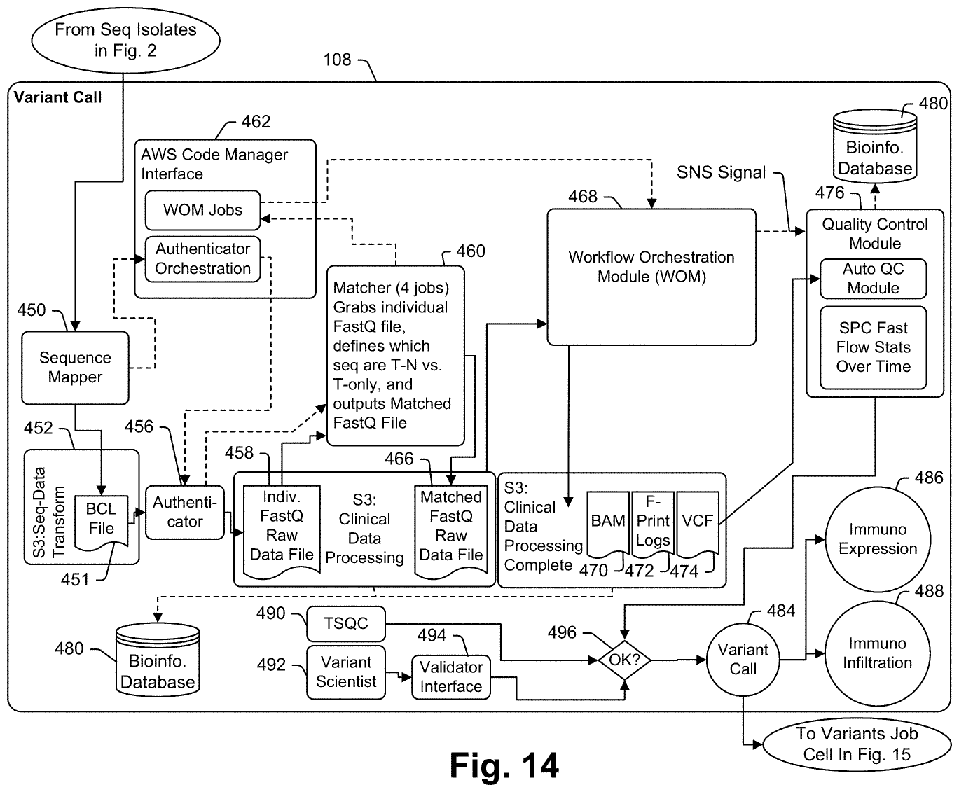

Referring once again to FIG. 2, order map 100 is presented in an extremely simple format in the interest of simplifying this explanation and each of the order sub-processes is fairly complex requiring activities and tasks by many different microservices and provider specialists. For example, the variant call sub-process 106 takes advantage of many different microservices to complete items 140 and 142 shown in FIG. 2. Referring now to FIG. 14, an exemplary more complex variant call and characterization sub-process is illustrated.

Referring to FIG. 2, after sequence data has been stored as data products by each of items 126, 130 and 132 and fulfillment IDs have been added to item specifications for each of those items, order hub 30 initiates the variant call sub-process 108. Referring also to FIG. 14, initially a sequencing mapper 450 accesses the sequencing data for a multiple sample (e.g., 55 samples, 45 samples with an additional 10 control samples for QC validation) workflow (hereinafter a “flowcell”) and mapper 450 maps subsets of the sequence data to specific samples to generate sample based raw sequencing data as a base call (BCL) file 451 that is stored in the AWS S3 cloud storage system (hereafter S3). An AWS code manager interface module 462 is a system that allows code to be deployed to AWS repositories for execution. The code manager 462 essentially validates system code is in good condition to run, deploys to AWS, monitors deployment until complete, and provides status info at each stage of deployment. More specific code manager tasks include setting up data dependencies for an authenticator, downloading data from AWS, authenticating sample sheets for each sequence result, indicates which sample is processed from which lane of the sequencer, identifies which files/indices are assigned to different samples, etc.

An authenticator module 456 converts the BCL file to a FASTQ file which is stored in S3. Module 456 also compares the raw sequencing data to sample sheets to determine, for each sample dataset, if the set is related to tumor-only or tumor-normal matched testing. Where a dataset is related to matched testing, module 456 calls on a Matcher module 460 to match the sequencing results from a tumor sample to the sequencing results from a normal sample of the same patient and stores a matched FASTQ file in S3 or similar cloud or internal data storage.

Referring again to FIG. 14, once sequencing data is in the FASTQ format and tumor-normal matching is complete, a workflow orchestration software module 468 initiates and manages a set of bioinformatics workflows which are executed for each genetic sequencing panel. (exemplary liquid biopsy NGS panel, exemplary whole exome NGS panel, exemplary solid tumor NGS panel, xG, etc.). In one embodiment the workflows occur in parallel and facilitate DNA Variant detection, DNA Fusion detection, and Metagenomics detection. Other characteristic detections are contemplated and will be added to the system as further developments occur. In general module 468 performs alignment, normalization, sorting and variant calling functions. Module 468 aligns DNA/RNA strands to begin identifying where they appear in the genome and then normalizes the data by removing duplicates corresponding to over amplified regions. Module 468 separates human from viral/bacterial/non-human data to ensure that only human DNA/RNA is processed. Next module 468 pairs/maps sequenced strands with a human reference genome and compares strands to identify variants in the tumor sample and/or to measure an abundance of at least one of the paired/mapped nucleotides.

An exemplary bioinformatics workflow may include the following. First, module 468 accesses the FASTQ file and runs an aligner software program (e.g., a Burrows-Wheeler Aligner (BWA)) to align a patient's sequence data and stores the results in a BAM file 470 in S3. Next, module 468 uses various software programs to call variants including, for instance, Freebayes and Pindel and in the case of exemplary liquid biopsy NGS panel, Vardict. Module 468 stores a Variant Call Format (VCF) file 474 in S3 with corresponding variant-allele-fraction (VAF) and coverage/equality metrics that are distinct from VAF. Module 468 filters out artifact noise and then uses CONA library & SNP-eff to identify one or more copy number variants (CNVs), single nucleotide polymorphisms (SNPs), insertions and deletions (InDels) and Fusions. Module 468 next generates fingerprint logs 468 memorializing DNA and RNA match as well as if tumor-normal and tumor-only match. Finally, module 468 formulates and transmits an SNS signal to indicate that variant calling is complete.

Referring still to FIG. 14, once module 468 generates and stores variant calls, a quality control module 476 initiates quality control processes to identify any irregularities in the VCF data results. Some quality control processes are automated. For instance, one simple automated process may check if matched tumor and normal data are both associated with the same gender. Another automated process may check if a variant was identified in all samples in a common workflow (e.g., all 55 samples in a flow) which would be highly irregular. At another instance, module 476 may track flowcell statistics over time to identify any irregularities or drift in results. Where quality is suspect, module 476 memorializes QC data in a bioinformatics database 480 and may initiate some corrective or notification process. As yet another instance, module 476 may check operational statistics including runtime, auto notification of delays, auto verification that data dependencies are satisfied when an “item complete” message is received, etc.

Referring still to FIG. 14, other quality control functions are contemplated like a trade standards QC module 490 that compares call results to standards within the industry and a manual process performed by a variant scientist 492 using a validator interface 494. Once at least a minimum set of quality controls are met (see decision block 496), the variant call process is complete as indicated by variant call item 484. Immuno expression and Immuno Infiltration items 486 and 488 are performed based on the variant call data products. At this point the variant call data is stored in a system database and the call sub-process is completed when a fulfillment ID is placed in the variant call item fulfillment field (see again FIG. 10). Once the variant call item is complete, order hub 30 publishes a notification indicating that the variant characterization sub-process 110 (see again FIG. 2) can be initiated.

In at least some embodiments order hub 30 maintains an audit log in database 34 that includes an order history. In general, each time any event occurs that is related to a system order, an event description referred to hereinafter as an “audit record” is stored within the audit log along with a timestamp indicating when the event occurred. The audit log is useful to analyze time series of changes that occur to an order. Several useful metrics can be extracted from the log data such as rates of exceptions within various systems, time to completion of each item and execution time distribution of order items. Order events that are tracked by the audit log include events that change the items tracked by order hub 30 such as (i) order creation and (i) order modification like adding an item to the order, cancelling an item from an order, adding an item sequence to support an additional test to an order, etc. In addition, events tracked by the log also include order and item status events such as item “in progress”, item “QC fail”, item “complete”, item “cancelled”, item “pause” and item “stop”. Other order and item related events may be memorialized in the log as well. In at least some cases the log will also include modifications to existing items within an order map (e.g., changing a physician's preferences related to a specific item).

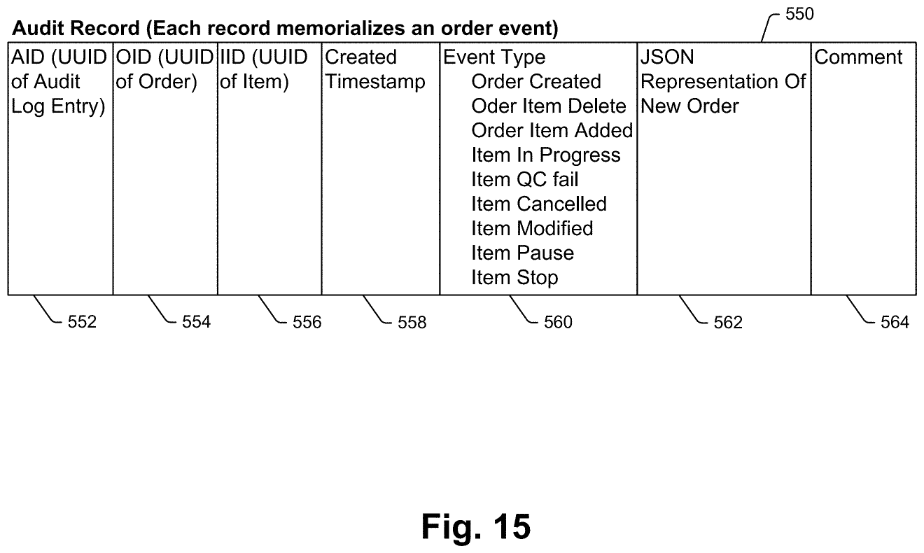

Referring now to FIG. 15, an exemplary audit record specification or data format 550 is illustrated that includes seven format fields including an audit ID field 552, an order ID field 554, an item ID field 556, a created timestamp 558, an event type field 560, a JSON field 562 and a comments field 564. A separate 4 character UUID that uniquely corresponds with a specific audit record is placed in field 552. Unique order and item UUIDs corresponding to an order and item that are affected by an event that is memorialized by an audit record are placed in fields 554 and 556, respectively. A time stamp corresponding to an event is placed in field 558. An event type is placed in field 560 and, as described above, may have any of several different values related to order item changes or item statuses.

A JSON in field 562 includes a representation of a new order after the event in field 560 has occurred. Here, the JSON reflects order item changes like new items added to an initial order and items deleted from an order that persist at the time indicated by the timestamp in field 558. In addition, in some cases the JSON may include information indicating the immediate status of each order item at the timestamped time including information indicating that an item is in progress, has been cancelled, has failed or is in a paused state. In other cases the JSON will not include all item status information and instead the system will access that information in other audit records corresponding to other order items when needed. Optional comments may be placed in field 562.

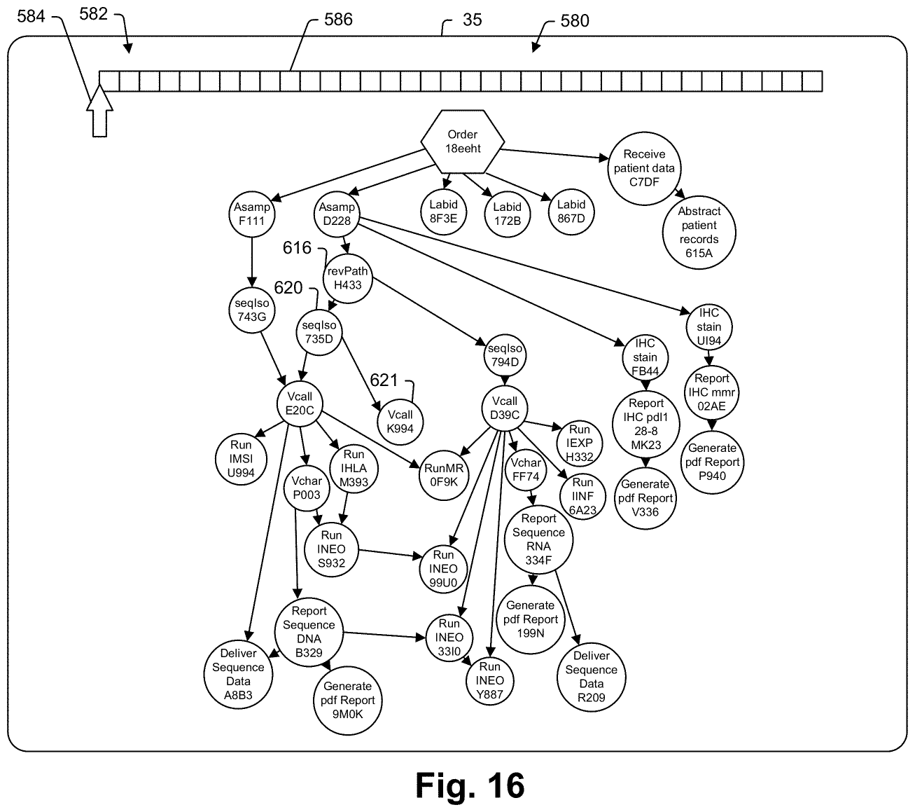

In addition to driving statistical analysis of various system operations, an order map and the audit record can be used to generate a detailed visual representation of a pending order or a completed order or a real time representation showing instantaneous status of an order currently in progress. To this end, see FIG. 16 that shows a screen shot 580 on an interface display screen 35 (see also FIG. 1) that shows a replication of the order map 200 from FIG. 3. A sliding control tool 582 includes a timeline 586 and a moveable pointer icon 584 that can be moved to different locations along the timeline 586 to select different points in time. After an order is stored in the order database 34 shown in FIG. 2, a system user may access a visual representation of that order as shown in FIG. 16 via display 35. The FIG. 16 map includes circular item representations that are all non-hatched meaning that none of the order items have been completed. The FIG. 16 representation corresponds to a time prior to order initiation when pointer icon 584 is far to the left on the timeline and no items are complete or even in-progress. Order hub 30 generates the FIG. 16 representation by converting a JSON file from an “order create” audit record into a DAG image.

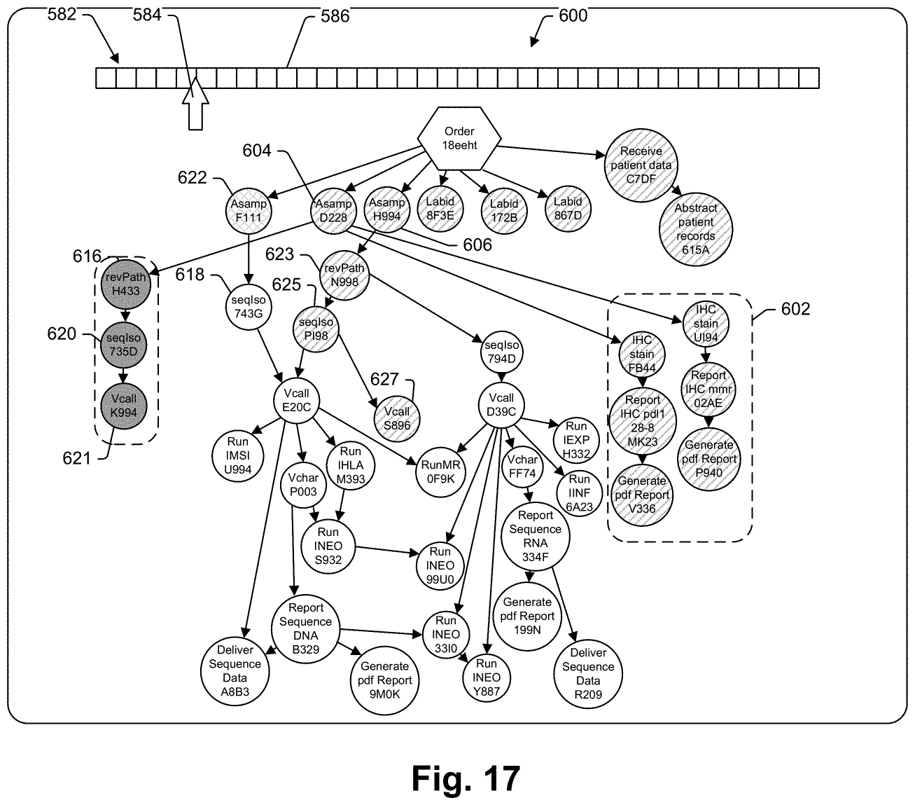

Referring to FIG. 17, a screenshot 600 similar to the screen shot 580 in FIG. 16 is shown, albeit where an associated order has commenced as indicated by the location of pointer icon 584 on timeline 586. In FIG. 17, many of the item icons are shown cross hatched left up to right, one 622 is shown double cross hatched ad three 616, 620 and 621 are shown shaded dark to indicate different item statuses. In FIG. 17 and subsequent figures, left up to right hatching will be used to indicate that an item has been completed and double diagonal cross hatching will be used to indicate that an item is currently in-progress. In addition, dark shading will be used to indicate that a quality control fail status has been assigned to an item and left down to right hatching will be used to indicate that an item has been cancelled. Thus, in FIG. 17, left up to right cross hatched items are completed, item 622 is in-progress and items 616, 620 and 621 have failed during some quality check.

Status map 600 is generated using the audit record that corresponds to an audit record timestamp that occurred just prior to the time selected on timeline 586, accessing the JSON representation of the order in the JSON field 562 associated with the identified audit record and converting the JSON representation to a DAG image as shown at 600. In cases where the JSON includes status information on all persisting order items, the JSON to DAG conversion can be direct. In cases where the JSON does not include all item statuses, order hub server 32 may access most recent audit records for each of the order map items that persists in the JSON to identify current statuses of each of those items and use that information to visually distinguish different item statuses on the DAG image.

Up to the time represented by the FIG. 17 DAG image what has happened is as follows. A tumor sample was received and processed. IHC PDL1 and MMR tests were completed (see box 602). At Vcall item 621 a tumor only preview was identified as incorrectly prepared for sequencing, and no sample from item 604 remained. Items 616, 620 and 621 were identified as QC fail (hence are shown as dark shaded) and a new sample accession item 606 was added to the order map along with new items 623, 625 and 627 to replicate the failed items 618, 620 and 621, respectively. The new sample was obtained and items 623, 625 and 627 have been successfully completed. Item 622 is in progress. IHC stains are of good quality, no change needed for those delivered tests.

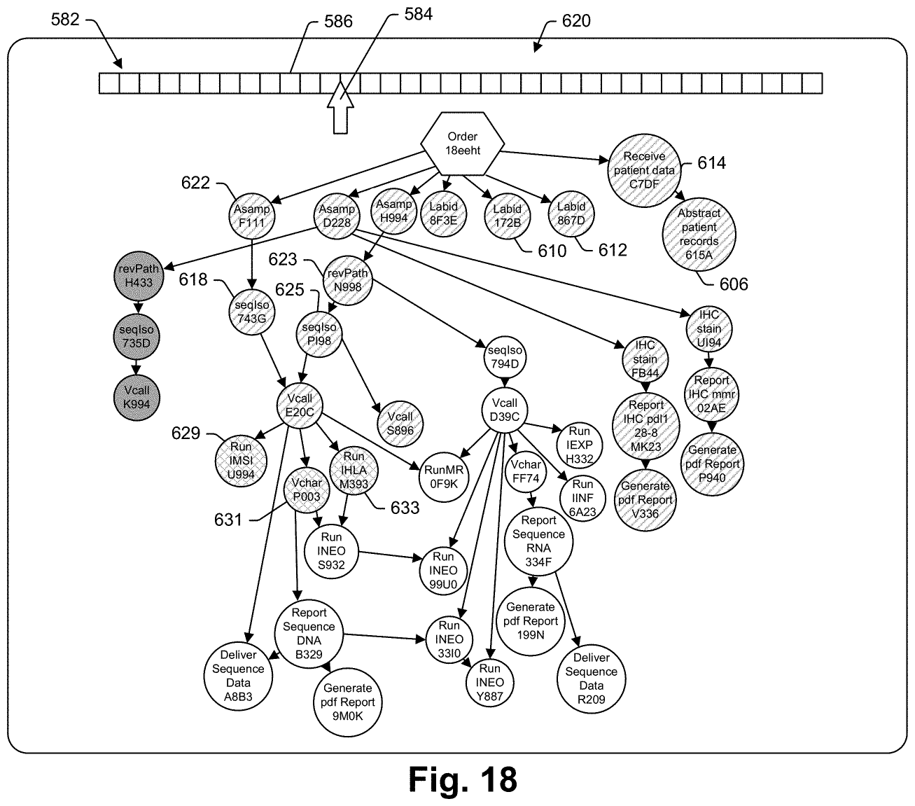

Referring to FIG. 18, a screen shot 620 similar to the screen shot 600 in FIG. 17 is shown, albeit where time has progressed further as indicated by the location of pointer icon 584 on timeline 586. In FIG. 18, many of the item icons are shown cross hatched left up to right indicating completion, several are shown shaded dark indicating QC fail status and items 629, 631 and 633 are double hatched indicating that each in progress. No further QC failures occurred as of the time shown in FIG. 18.

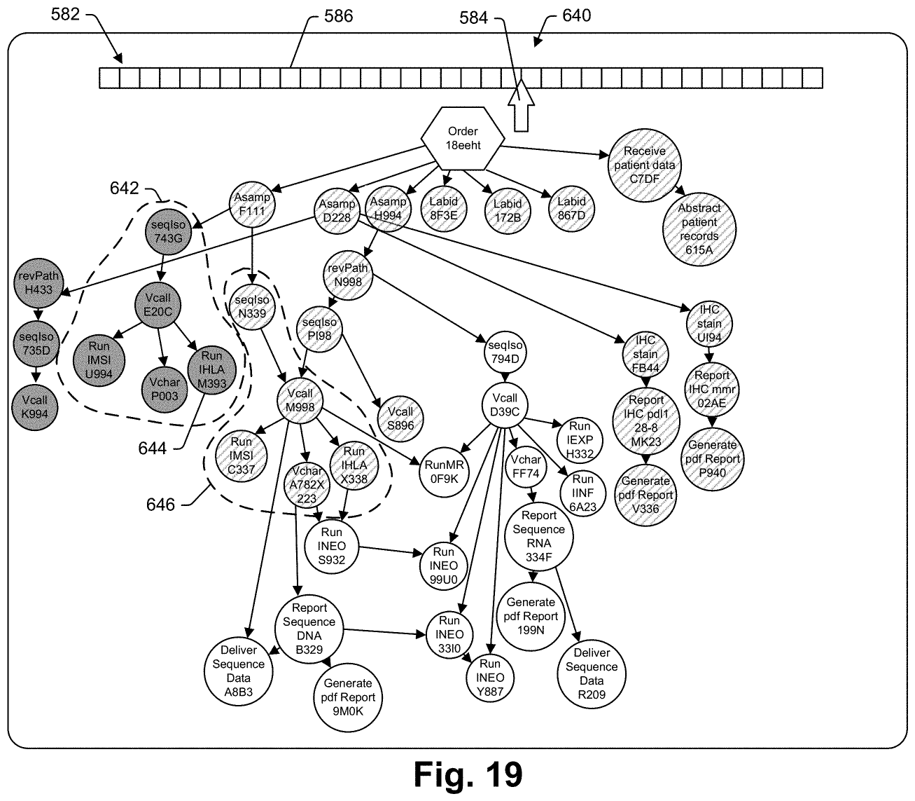

Referring to FIG. 19, a screen shot 640 similar to the screen shot 620 in FIG. 18 is shown, albeit where time has progressed further as indicated by the location of pointer icon 584 on timeline 586. Up to the time represented by the FIG. 19 DAG image what has happened is as follows. Items in set 642 excluding item 644 were completed at time 584 when item 644 issues were identified with the Normal sample sequencing output and therefore the normal sample sequencing had to be re-executed. To cause re-execution, items in set 646 were added to the order map to replace items in set 642. The normal sample sequence was completed.

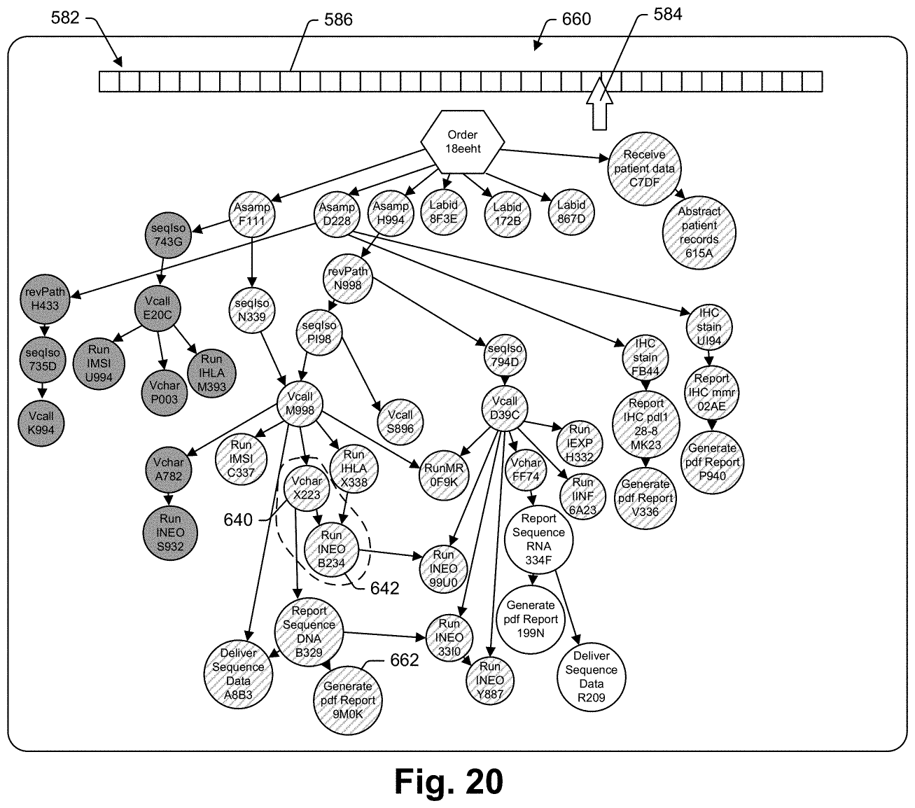

Referring to FIG. 20, a screen shot 660 similar to the screen shot 640 in FIG. 19 is shown, albeit where time has progressed further as indicated by the location of pointer icon 584 on timeline 586. Up to the time represented by the FIG. 20 DAG image what has happened is as follows. An AI variant science auto-categorization program was run which generated a notification requiring manual review. Manual variant science was performed and a report signed out as indicated by item 662. The deliver sequence data item at 664 encountered an error so that item was temporarily delayed (not shown in Figs. but “delay” status would be shown visually distinguished from other DAG image statuses). A provider specialist identified the source of the item 664 error, corrected it, and restarted the item 664 which then completed successfully. Most of the other items in the order are shown as complete. RNA sequencing commenced once DNA tests were complete.

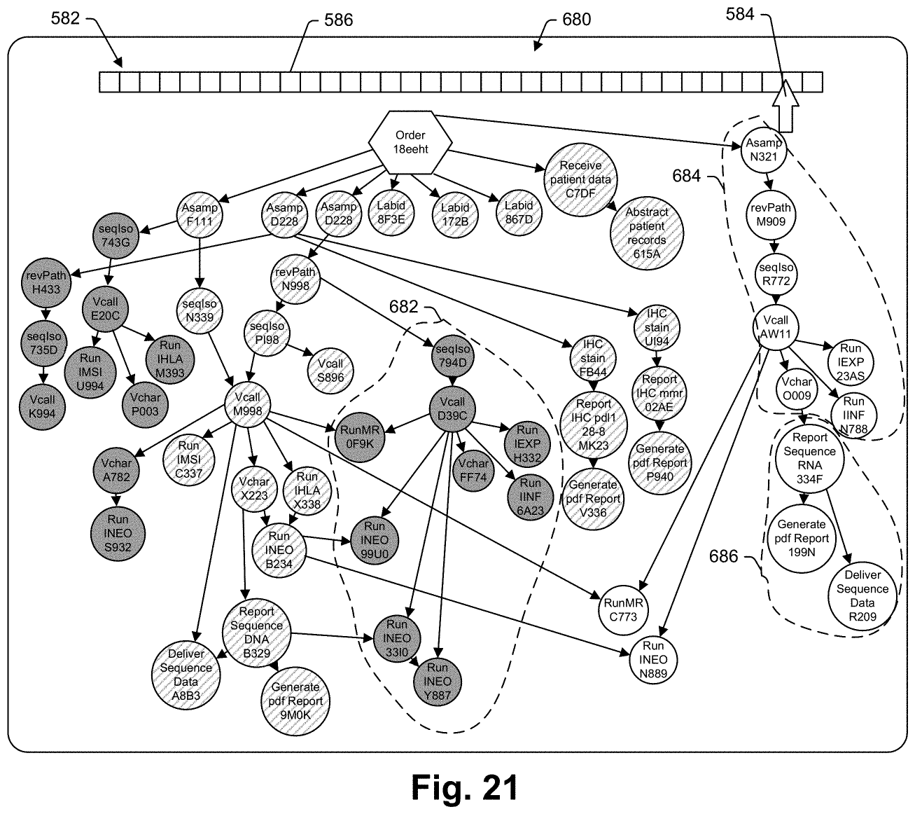

Referring to FIG. 21, a screen shot 680 similar to the screen shot 6460 in FIG. 20 is shown, albeit where time has progressed further as indicated by the location of pointer icon 584 on timeline 586. Up to the time represented by the FIG. 20 DAG image what has happened is as follows. After many RNA items were completed (see again FIG. 19), report review identified that an inadvertent sample swap occurred during RNA sequencing in the lab, and no additional sample remains. Again, a new sample is required and the RNA testing items have to be re-executed. To this end, prior completed RNA items in set 682 are all set to a QC fail status (as indicated by the dark shading in the DGA image) and a new set of RNA test items 684 is added to the order map starting with a new sample accession item. The original report sequence RNA, Generate pdf Report and Deliver Sequence Data items in set 686 remain and are simply fitted onto the new RNA test set 684. The DNA report from the same sample is unaffected by the RNA swap. After the time corresponding to the FIG. 21 DAG image it is assumed the remainder of the order processes without any QC fail issues.

While the invention may be susceptible to various modifications and alternative forms, specific embodiments have been shown by way of example in the drawings and have been described in detail herein. However, it should be understood that the invention is not intended to be limited to the particular forms disclosed. For example, in cases where report worthy information becomes available after a report is signed out, the disclosed system may support a report addendum process whereby a prior completed order is reopened and additional items are added to the order map to access and consume the new information and the system may then generate an updated report accordingly. For example, while the systems described above are described in the context of a system where samples need to accessioned and processed, in other cases it is contemplated that a physician or a patient may have her own sequencer at home or at a clinic and may send in a VCL file from a personal sequencer instead of a tissue sample. In these cases, an order would not include accessioning sample and other similar items and instead would start with items that assume sequencing is complete. Thus, the exemplary order system would be able to start at any point in a testing, analysis and reporting process and should be able to operate in the manner described above.

In addition, in at least some cases it is contemplated that the above system could be used to manage other complex medical order processes, patient treatments or clinical activities, orders related to other disease states, etc.

Thus, the invention is to cover all modifications, equivalents, and alternatives falling within the spirit and scope of the invention as defined by the following appended claims.

To apprise the public of the scope of this invention, the following claims are made:

APPENDIX A
Table 1-Non-Exhaustive Set Of Item Types and Related
Information

| | | | | | Additional Required |
|---|---|---|---|---|---|
| Item | | | | | Item Data (Based on |
| Type | Description | Fulfillment | Depend | Data Format | ItemType |
| (1) Receive- | Tracks | Links | None | { | NA |
| patient- | receipt | to a | | “id”: “<id>” | |
| records | of | patient id | | “type”: “receive- | |
| | clinical | | | patient-records”, | |
| | documents | | | “fulfillment”: { | |
| | for a | | | “type”: “patient”, | |
| | patient. | | | “id”: “<value>” | |
| | | | | }, | |
| | | | | “status”: null, | |
| | | | | “dependencies”: | |
| | | | | [ ] | |
| | | | | } | |
| (2) Abstract- | Tracks | Links | One | { | NA |
| patient | completion of | to a | patient- | “id”: “<id>” | |
| | extracting | patient id | records- | “type”: “abstract- | |
| | details | | received | patient”, | |
| | about a | | item | “fulfillment”: { | |
| | patient | | | “type”: “patient”, | |
| | from | | | “id”: “<value>” | |
| | clinical | | | }, | |
| | documents. | | | “status”: null, | |
| | | | | “dependencies”: | |
| | | | | [ | |
| | | | | “<receive- | |
| | | | | patient-records- | |
| | | | | item-id>” | |
| | | | | ] | |
| | | | | } | |
| | Tracks | Must | None | { | TissueClassification- |
| (3) Accession- | the | link to | | “id”: “<id>” | This indicate |
| sample | receipt | a | | “type”: | the type of |
| | of a | record for | | “accession- | tissue to be received. |
| | physical | saple | | sample”, | Possible values: |
| | specimen | | | “fulfillment”: { | (tumor, |
| | from a | | | “type”: | normal) |
| | patient | | | “specimen”, | TissueSource-This |
| | and | | | “id”: “<value>” | indicate the type of |
| | physician. | | | }, | tissue to be received. |
| | System | | | “status”: null, | Possible values: |
| | may | | | “dependencies”: | (human, |
| | also | | | [ ], | Mouse, organoid) |
| | receive | | | “tissueClassification”: | SlideCount-Indicates |
| | a | | | “tumor”, | the number of slides to |
| | substance | | | “tissueSource”: | receive. Integer value |
| | refined | | | “human”, | (default 0) |
| | from a | | | “slideCount”: 0, | SlideStain-This |
| | tissue or | | | “slideStain”: null | indicate the type of |
| | fluid | | | } | stain to be received. |
| | specimen--an | | | | Possible values: (null |
| | Isolate. | | | | (default), H&E) |
| (4) Review- | Tracks | Must | One | { | NA |
| pathology | completion | link to | accession- | “id”: “<id>” | |
| | of | one | sample | “type”: “review- | |
| | Path | pathology | item | pathology”, | |
| | Review | review | | “fulfillment”: { | |
| | for a | experiment | | “type”: | |
| | tumor | ID | | “pathology-analysis”, | |
| | sample. | | | “id”: “<value>” | |
| | | | | }, | |
| | | | | “status”: null, | |
| | | | | “dependencies”: | |
| | | | | [ | |
| | | | | “<accession- | |
| | | | | sample-item-id>” | |
| | | | | ] | |
| | | | | } | |
| (5) | Tracks | Must | One | { | Assay-Assay |
| Sequence | that | link to | accession- | “id”: “<id>” | for which the |
| isolate | sequencing of a | sequenced | sample | “type”: “sequence- | isolate is to |
| | patient's | asset | item OR | isolate | be prepared. |
| | sample | ID | One | “fulfillment”: { | Possible values |
| | is | | review- | “type”: | (whole |
| | imminent. | | pathology | “sequencer- | exome, |
| | An | | item | isolate”, | solid tumor, |
| | Isolate | | | “id”: “<value>” | liquid biopsy. |
| | is | | | }, | Analyte-Analyte |
| | prepared | | | “status”: null, | variation |
| | from a | | | “dependencies”: [ | for which the |
| | patent's | | | “<accession- | isolate is to be |
| | sample | | | sample-item-id>” | prepared. Possible |
| | for a | | | ], | values |
| | specific | | | “assay”: “whole- | (RNA, DNA). |
| | genomic | | | exome”, | Coverage-The |
| | panel | | | “analyte”: “dna” | variability in |
| | and | | | } | coverage as an |
| | analyte | | | | option for |
| | (rna, | | | | RNA and DNA. |
| | dna) | | | | Possible |
| | combination | | | | values (low, high. |
| | and is | | | | RelativePriority- |
| | placed | | | | The |
| | in a | | | | relative |
| | Flow | | | | priority of this |
| | Cell | | | | sequence isolate |
| | destined | | | | (e.g., 1 2 3 . . . N). |
| | for a | | | | |
| | genomic | | | | |
| | sequencer. | | | | |
| (6) | Tracks | Database | One | { | bucketname-The |
| Deliver- | the | url | analyze- | “id”: “<id>” | database |
| sequence- | action | for | variant-call | “type”: “deliver- | bucket where |
| data | of | delivered | item | sequence-data”, | data should be |
| | copying | content | (research | “fulfillment”: { | stored. |
| | raw | | flow) OR | “type”: “sequence- | |
| | sequence | | One | data”, | |
| | data to | | deliver- | “id”: “<value>” | |
| | a | | report item | }, | |
| | partner | | (clinical | “status”: null, | |
| | institution's | | flow) | “dependencies”: [ | |
| | System | | | “<analyze-variant- | |
| | database | | | call>” | |
| | bucket. | | | ], | |
| | | | | “bucketname”: | |
| | | | | “<value>” | |
| | | | | } | |
| (7) | Tracks | Must | One or Two | { | detectF'usions- |
| Analyze- | completion | link to | sequence- | “id”: “<id>” | Indicates |
| variant- | of | one | isolate | “type”: “analyze- | whether to |
| call | the | bioinfo. | Items. If | variant-call”, | detect fusions. |
| | pipeline | Analysis ID | two, both | “fulfillment”: { | |
| | that is | | items must | “type”: “variant- | |
| | managed | | have the | call”, | |
| | by the | | same | “id”: “<value>” | |
| | Bioinformatics | | values for | }, | |
| | team. | | ‘assay’ and | “status”: null, | |
| | Variant | | ‘analyte’ | “dependencies”: [ | |
| | calling | | analysis ID | “<sequence- | |
| | is | | | isolate-item-id>”, | |
| | completed | | | ... | |
| | using | | | ], | |
| | the | | | “detectFusions”: | |
| | sequencer | | | false | |
| | output | | | } | |
| | of one | | | | |
| | or two | | | | |
| | Isolates. | | | | |
| (8) | Tracks | Must | One | { | NA |
| Analyze- | completion | link to | analyze- | “id”: “<id>” | |
| variant- | of a | one | variant-call | “type”: “analyze- | |
| char. | variant | record | Item | variant- | |
| | characterization | for a | | characterization”, | |
| | analysis | variant | | “fulfillment”: { | |
| | that is | char. | | “type”: | |
| | completed | analysis | | “[primary\|secondary]- | |
| | by | in the | | variant- | |
| | Variant | form | | characterization\|rn | |
| | Science | of a | | a-expression-calls”, | |
| | and the | database | | “id”: “<value>” | |
| | P | object | | }, | |
| | product. | key | | “status”: null, | |
| | Variant | | | “dependencies”: [ | |
| | characterization | | | “<abstract-patient- | |
| | is | | | item-id>”, | |
| | completed | | | “<analyze-variant- | |
| | using | | | call-item-id>” | |
| | the | | | ] | |
| | variant | | | } | |
| | calls | | | | |
| | that are | | | | |
| | produced | | | | |
| | by the | | | | |
| | Bioinfo. | | | | |
| | pipeline | | | | |
| (9) Select- | Tracks | Must | One | { | |
| therapies | completion of | link to | analyze- | “id”: “<id>” | |
| | recommendations | one | variant- | “type”: “select- | |
| | of | record | characterization | therapies”, | |
| | therapies. | for a | item | “fulfillment”: { | |
| | | therapy | | “type”: “therapy- | |
| | | selection | | match”, | |
| | | in | | “id”: “<value>” | |
| | | the | | }, | |
| | | form | | “status”: null, | |
| | | of a | | “dependencies”: [ | |
| | | database | | “<analyze-variant- | |
| | | object | | characterization- | |
| | | key | | item-id>” | |
| | | | | ] | |
| | | | | } | |
| (10) Run- | T tracks | Must | analyze- | { | NA |
| immuno-hla | completion | link to | variant-call | “id”: “<id>” | |
| | the | of one | | “type”: “run- | |
| | HLA | record | | immuno-hla”, | |
| | immunotherapy | for | | “fulfillment”: { | |
| | module. | HLA | | “type”: “immuno-hla”, | |
| | | immunotherapy | | “id”: “<value>” | |
| | | data | | }, | |
| | | in the | | “status”: null, | |
| | | form | | “dependencies”: [ | |
| | | of | | “<analyze-variant- | |
| | | database | | call>”, | |
| | | object | | | |
| | | key | | | |
| (11) Run- | Tracks | Must | analyze- | { | |
| immuno-msi | completion | link to | variant-call | “id”: “<id>”, | |
| | of | one | | “type”: “run- | |
| | the MSI | record | | immuno-msi”, | |
| | immuno | for | | “fulfillment”: { | |
| | therapy | MSI | | “type”: “immuno-msi”, | |
| | module. | immunotherapy | | “id”: “<value>” | |
| | | data | | }, | |
| | | in the | | “status”: null, | |
| | | form | | “dependencies”: [ | |
| | | of a | | “<analyze-variant- | |
| | | database | | call>”, | |
| | | object | | | |
| | | key | | | |
| (12) Run- | Tracks | Must | analyze- | { | |
| immuno- | completion | link to | variant-call | “id”: “<id>”, | |
| infiltration | of | one | | “type”: “run- | |
| | the | record | | immuno-infiltration”, | |
| | Infiltration | for | | “fulfillment”: { | |
| | immuno | infiltration | | “type”: | |
| | therapy | data in | | “immuno-infiltration”, | |
| | module. | the | | “id”: “<value>” | |
| | | form | | }, | |
| | | of a | | “status”: null, | |
| | | database | | “dependencies”: [ | |
| | | object | | “<analyze-variant-call>”, | |
| | | key | | | |
| (13) | Tracks | Must | analyze- | { | |
| Run- | completion | link to one | variant-call | “id”: “<id>” | |
| immuno- | of the | record | | “type”: “run-immuno- | |
| expression- | Expression | for | | expression-targets”, | |
| targets | Targets | expression | | “fulfillment”: { | |
| | immuno | targets | | “type”: “immuno- | |
| | therapy | immunotherapy | | expression- | |
| | module. | data | | targets”, | |
| | | in the | | “id”: “<value>” | |
| | | form | | }, | |
| | | of | | “status”: null, | |
| | | A | | “dependencies”: [ | |
| | | database | | “<analyze variant | |
| | | object | | call>”, | |
| | | key | | | |
| (14) Run- | Tracks | Must | report- | { | |
| immuno- | completion | link to | sequence- | “id”: “<id>” | |
| neoantigen | of the | one | dna | “type”: “run- | |
| | Neoantigen | record | | immuno-neoantigen”, | |
| | immuno | for | | “fulfillment”: { | |
| | therapy | neoantigen | | “type”: “immuno- | |
| | module. | immunotherapy | | neoantigen”, | |
| | | data | | “id”: “<value>” | |
| | | in the | | }, | |
| | | form | | “status”: null, | |
| | | of a | | “dependencies”: [ | |
| | | database | | “<report-sequence-dna>”, | |
| | | object | | | |
| | | key | | | |
| (15) Match- | Tracks | Must | One | { | |
| clinical- | completion | link to | analyze- | “id”: “<id>” | |
| trials | recommendations | one | variant- | “type”: “match- | |
| | of | record | characterization | clinical-trials”, | |
| | clinical | for a | item | “fulfillment”: { | |
| | trials. | clinical | | “type”: “clinical- | |
| | | trials | | trials-match”, | |
| | | matching | | “id”: “<value>” | |
| | | in | | }, | |
| | | the | | “status”: null, | |
| | | form | | “dependencies”: [ | |
| | | of a | | “<analyze-variant- | |
| | | database | | characterization- | |
| | | object | | item-id>”, | |
| | | key | | ] | |
| | | | | } | |
| (16) IHC- | Tracks | Must | One | { | Stain-The type |
| stain | completion | link to | accession- | “id”: “<id>” | of stain to |
| | of | one | sample | “type”: “ihc-stain”, | use. Possible values: |
| | the | IHC | item | “fulfillment”: { | pd-11-28-8 |
| | staining | review | | “type”: “ihc-stain”, | mmr |
| | of slides | experiment | | “id”: “<value>” | c-met |
| | for an | ID | | }, | axl |
| | IHC | | | “status”: null, | cd73 |
| | report, | | | “dependencies”: [ | arginase |
| | scanning | | | “<accession- | her2 |
| | and | | | sample-item-id>” | cd166 |
| | uploading | | | ], | Slide Count-The |
| | the | | | stain: “pdl1-28-8”, | number of |
| | slide, | | | slideCount: 4 | slides to stain. |
| | and path | | | } | Value is an |
| | review | | | | integer |
| | of the | | | | |
| | slide. | | | | |
| (17) Report- | Tracks | Must | One | { | |
| sequence- | completion | link to | abstract- | “id”: “<id>” | |
| dna | and | one | patient item | “type”: “report- | |
| | sign-out | DNA | One | sequence-dna”, | |
| | of a | report | analyze- | “fulfillment”: { | |
| | DNA | ID | variant-call | “type”: “dna- | |
| | sequencing | | item for | sequence-report”, | |
| | report. | | DNA | “id”: “<value>” | |
| | | | samples | }, | |
| | | | One | “status”: null, | |
| | | | analyze- | “dependencies”: [ | |
| | | | variant- | “<abstract-patient- | |
| | | | characterization | item-id>”, | |
| | | | item | “<analyze-variant- | |
| | | | linked to | call-item-id>”, | |
| | | | previous | “<variant- | |
| | | | analyze- | characterization- | |
| | | | variant-call | analysis-item-id>”, | |
| | | | One | “<clinical-therapy- | |
| | | | clinical- | match-item-id>”, | |
| | | | therapy- | ], | |
| | | | match item | } | |
| | | | linked to | | |
| | | | previous | | |
| | | | analyze- | | |
| | | | variant- | | |
| | | | characterization | | |
| (18) Report- | Tracks | Must | One | | |
| sequence-rna | completion | link to | analyze- | | |
| | and | one | variant-call | | |
| | sign-out | record | item for | | |
| | of a | for an | RNA | | |
| | RNA | RNA | samples | | |
| | sequencing | report | | | |
| | report. | | | | |
| (19) Report- | Tracks | Must | One | | |
| sequence-qns | completion | link to | accession- | | |
| | and | one | sample or | | |
| | sign-out | record | analyze- | | |
| | of a | for an | variant-call | | |
| | Quality | QNS | item that is | | |
| | Not | report | in qc-fail | | |
| | Sufficient | | status | | |
| | report. | | | | |
| (20) Report- | Tracks | Links | ihc-stain | | |
| ihc- | completion | to one | | | |
| mmr | and | record | | | |
| | sign-out | for an | | | |
| | of an | MMR | | | |
| | Immuno- | IHC | | | |
| | histochemistry | report | | | |
| | Report | | | | |
| | for | | | | |
| | Mismatch | | | | |
| | Repair | | | | |
| | detection. | | | | |
| (21) Report- | Tracks | Links | ihc-stain | | |
| ihc- | completion | to one | | | |
| pdl1- | and | record | | | |
| 22c3 | sign-out | for an | | | |
| | of an | PDL1 | | | |
| | Immuno- | 22c3 | | | |
| | histochemistry | IHC | | | |
| | Report | report | | | |
| | using | | | | |
| | Dako | | | | |
| | PDL1 | | | | |
| | 22c3 | | | | |
| | Stain. | | | | |
| (22) Report- | Tracks | Links | ihc-stain | | |
| ihc- | completion | to one | | | |
| pdl1- | and | record | | | |
| 28-8 | sign-out | for an | | | |
| | of an | PDL1 | | | |
| | Immuno- | 28-8 | | | |
| | histochemistry | report | | | |
| | Report | | | | |
| | using | | | | |
| | PDL1 | | | | |
| | 28-8 | | | | |
| | Stain. | | | | |
| (23) Report- | Tracks | Must | One of any | | |
| amendment | completion | link to | report item | | |
| | and | one | | | |
| | sign-out | record | | | |
| | of an | for an | | | |
| | Amendment | Amendment | | | |
| | (correction) | report | | | |
| | report. | | | | |
| (24) Report- | Tracks | Must | One of any | | |
| addendum | completion | link to | report item | | |
| | and | one | | | |
| | sign-out | record | | | |
| | of an | for an | | | |
| | Addendum | Addendum | | | |
| | (additional | report | | | |
| | information) | | | | |
| | report. | | | | |
| (25) | Tracks | ID of | One Report | { | |
| Generate- | completion | the | Review | “id”: “<id>” | |
| pdf | of | report | item | “type”: “generate- | |
| | PDF | in | | pdf-report, | |
| | generation | Attachments | | “fulfillment”: { | |
| | and | Service | | “type”: “pdf- | |
| | upload | | | report-generate”, | |
| | to | | | “id”: “<value>” | |
| | Attachments | | | }, | |
| | Service. | | | “status”: null, | |
| | | | | “dependencies”: [ | |
| | | | | “<report-[report | |
| | | | | type]>”, | |
| | | | | ] | |
| | | | | } | |
| (26) Run- | Tracks | Must | One report- | { | |
| cohort | completion | link to | sequence- | “id”: “<id>” | |
| | of | one | dna item | “type”: “run-cohort”, | |
| | running | report | | “fulfillment”: { | |
| | the | ID | | “type”: “cohort- | |
| | cohort | | | run”, | |
| | job. | | | “id”: “<value>” | |
| | | | | }, | |
| | | | | “status”: null, | |
| | | | | “dependencies”: [ | |
| | | | | “<report- | |
| | | | | sequence-dna>”, | |
| | | | | ] | |
| | | | | } | |
| (27) Deliver- | Tracks | Must | Generate | | |
| report | the | link to | PDF Item | | |
| | status of | one | | | |
| | delivery | record | | | |
| | or a | for a | | | |
| | PDF | Report | | | |
| | report. | delivered | | | |
| (28) Report- | Tracks | Must | None | { | |
| gen-lab- | linking | link to | | “id”: “<id>” | |
| order | of an | a X lab | | “type”: “report- | |
| | order | order | | gen-lab-order”, | |
| | hub | id | | “fulfillment”: { | |
| | order to | | | “type”: “report-gen- | |
| | a | | | lab-order-id”, | |
| | service | | | “id”: “<value>” | |
| | request. | | | }, | |
| | | | | “status”: null, | |
| | | | | “dependencies”: [ ] | |
| | | | | } | |
| (29) Receive | Tracks | Must | None | { | Analyte-The analyte |
| external- | the | link to | | “id”: “<id>” | variation for which |
| sequencing- | expected | a | | “type”: “external- | the isolate is |
| data | receipt | Manifest | | data, | to be prepared |
| | and | Database | | “fulfillment”: { | (RNA or DNA). |
| | fulfilment | File, | | “type”: “data-file- | Assay-The |
| | of | | | receipt”, | System external |
| | sequencing | | | “id”: “<value>” | assay for which |
| | data | | | }, | the isolate is |
| | files | | | “status”: null, | to be prepared. |
| | provided | | | “dependencies”: [ ], | TissueClassification- |
| | by a lab | | | “analyte”: “dna” | Indicate the |
| | external | | | “panel”: | classification of |
| | to | | | “uci.archer.variant | sequenced tissue |
| | System. | | | plex.core- | as tumor or |
| | An | | | myeloid”, | normal. |
| | Isolate | | | “tissueClassification”: | TissueSource- |
| | has | | | “tumor”, | Indicate the |
| | been | | | “tissueSource”: | source of the |
| | prepared | | | “human”, | sequenced |
| | and | | | } | tissue as human, |
| | sequenced | | | | mouse, or |
| | from | | | | organoid |
| | a | | | | |
| | patent's | | | | |
| | sample | | | | |
| | for this | | | | |
| | lab's | | | | |
| | specific | | | | |
| | panel | | | | |
| | and a | | | | |
| | raw data | | | | |
| | file is | | | | |
| | sent to | | | | |
| | System. | | | | |

What is claimed is:

1. A genomic test processing system comprising:
an order management engine;
one or more order processing engines, comprising:
a receiving engine, to receive a state of an order from the order management engine;
an execution engine, to:
determine a sequence of steps to advance the received state of an order to a final state;
iteratively designate each step of the sequence of steps as completed before initiating the next step of the sequence of steps; and
advance the state of the order to a final state when a last step of the sequence of steps is completed; and
a broadcasting engine, to broadcast the final state of the order to the order management engine; and
wherein the order management engine causes one of the one or more order processing engines to generate a next-generation sequencing report from the final state of the order.

2. The system of claim 1, wherein the received state of an order indicates DNA processing of a specimen, the sequence of steps further comprising:
scraping a prepared FFPE slide to collect a sample of the specimen's tissue;
isolating DNA nucleotides from the sample;
amplifying the isolated nucleotides;
sequencing the amplified nucleotides;
mapping the sequenced nucleotides to a human reference genome;
identifying genetic variants from the human reference genome in the sequenced nucleotides; and
generating a report from the identified genetic variants.

3. The system of claim 1, wherein the received state of an order indicates RNA processing of a specimen, the sequence of steps further comprising:
scraping a prepared FFPE slide to collect a sample of the specimen's tissue;
isolating RNA nucleotides from the sample;
amplifying the isolated nucleotides;
sequencing the amplified nucleotides;
mapping the sequenced nucleotides to a human reference genome;
measuring an abundance of at least one of the mapped nucleotides; and
generating a report from the measured abundance of the at least one of the mapped nucleotides.

4. The system of claim 1, wherein a first order processing engine receives the state of an order indicating DNA processing of a specimen and a second order processing engine receives the state of the order indicating RNA processing of the specimen, further comprising:
at the first order processing engine:
scraping a prepared FFPE slide to collect a first sample of the specimen's tissue;
isolating DNA nucleotides from the first sample;
amplifying the isolated DNA nucleotides;
sequencing the amplified nucleotides;
mapping the sequenced nucleotides to a human reference genome;
identifying genetic variants from the human reference genome in the sequenced nucleotides; and
generating a report from the identified genetic variants; and
at the second order processing engine:
scraping a prepared FFPE slide to collect a second sample of the specimen's tissue;
isolating RNA nucleotides from the second sample;
amplifying the isolated RNA nucleotides;
sequencing the amplified nucleotides;
mapping the sequenced nucleotides to a human reference genome;
measuring an abundance of at least one of the mapped nucleotides; and
generating a report from the measured abundance of the at least one of the mapped nucleotides; and
wherein the first and the second order processing engines operate concurrently.

5. The system of claim 2, wherein:
one or more of the following steps occur at a first geographic location:
scraping a prepared FFPE slide to collect a sample of the specimen's tissue;
isolating DNA nucleotides from the sample;
amplifying the isolated nucleotides; and
sequencing the amplified nucleotides; and
one or more of the following steps occur in a cloud-based architecture:
mapping the sequenced nucleotide to a human reference genome;
identifying genetic variants from the human reference genome in the sequenced nucleotides; and
generating a report from the identified genetic variants.

6. The system of claim 2, wherein:
one or more of the following steps occur at a first geographic location:
scraping a prepared FFPE slide to collect a sample of the specimen's tissue;
isolating RNA nucleotides from the sample;
amplifying the isolated nucleotides; and
sequencing the amplified nucleotides; and
one or more of the following steps occur at a second geographic location:
mapping the sequenced nucleotides to a human reference genome;
measuring an abundance of at least one of the mapped nucleotides; and
generating a report from the measured abundance of the at least one of the mapped nucleotides.

7. The system of claim 5, wherein the report from the identified genetic variants also comprises information based on a database of information of individuals with a health condition similar to that of a patient who was a source of the specimen.

8. The system of claim 6, wherein the report from the measured abundance of the at least one of the mapped nucleotides also comprises information based on a database of information of individuals with a health condition similar to that of a patient who was a source of the specimen.

9. The system of claim 1, wherein the genomic test includes tumor-normal sequencing, and wherein a first order processing engine receives the state of an order indicating DNA processing of a normal specimen and a second order processing engine receives the state of the order indicating DNA processing of a tumor specimen, further comprising:
at the first order processing engine:
scraping a prepared FFPE slide to collect a sample of the normal specimen's tissue;
isolating normal DNA nucleotides from the normal sample;
amplifying the isolated normal nucleotides;
sequencing the amplified normal nucleotides;
mapping the sequenced normal nucleotides to a human reference genome; and
identifying genetic variants of the normal sample from the human reference genome in the sequenced nucleotides;
at the second order processing engine:
scraping a prepared FFPE slide to collect a sample of the tumor specimen's tissue;
isolating tumor DNA nucleotides from the tumor sample;
amplifying the isolated tumor nucleotides;
sequencing the amplified tumor nucleotides;
mapping the sequenced tumor nucleotides to a human reference genome;
identifying genetic variants of the tumor sample from the human reference genome in the sequenced nucleotides; and
generating a report from the identified genetic variants of the tumor sample based at least in part on the identified genetic variants of the normal sample.

10. The system of claim 9, wherein:
one or more of the following steps occur at a first geographic location:
scraping a prepared FFPE slide to collect a sample of the normal specimen's tissue;
isolating normal DNA nucleotides from the normal sample;
amplifying the isolated normal nucleotides;
sequencing the amplified normal nucleotides;
scraping a prepared FFPE slide to collect a sample of the tumor specimen's tissue;
isolating tumor DNA nucleotides from the tumor sample;
amplifying the isolated tumor nucleotides;
sequencing the amplified tumor nucleotides; and
one or more of the following steps occur in a cloud-based architecture:
mapping the sequenced tumor nucleotides to a human reference genome;
mapping the sequenced normal nucleotides to a human reference genome;
identifying genetic variants of the tumor sample from the human reference genome in the sequenced nucleotides;
identifying genetic variants of the normal sample from the human reference genome in the sequenced nucleotides; and
generating a report from the identified genetic variants of the tumor sample based at least in part on the identified genetic variants of the normal sample.

11. The system of claim 1, wherein the received state of an order indicates DNA processing of a liquid biopsy specimen, the sequence of steps further comprising:
removing cells from the liquid biopsy specimen to collect a cell-free sample of the specimen;
isolating DNA nucleotides from the cell-free sample;
amplifying the isolated nucleotides;
sequencing the amplified nucleotides;
mapping the sequenced nucleotides to a human reference genome;
identifying genetic variants from the human reference genome in the sequenced nucleotides; and
generating a report from the identified genetic variants.

12. The system of claim 1, wherein the received state of an order indicates DNA processing of a specimen, the sequence of steps further comprising:
scraping a prepared FFPE slide to collect a sample of the specimen's tissue;
isolating DNA nucleotides from the sample;
amplifying the isolated nucleotides;
sequencing the amplified nucleotides;
mapping the sequenced nucleotides to a human reference genome;
identifying genetic variants from the human reference genome in the sequenced nucleotides;
generating a tumor mutational burden (TMB) score;
generating a microsatellite instability (MSI) score; and
generating a report from the identified genetic variants, TMB score, and MSI score.

13. The system of claim 1, wherein the received state of an order indicates DNA processing of a blood specimen, the sequence of steps further comprising:
extracting peripheral whole blood to collect a sample of the specimen;
isolating DNA nucleotides from the sample;
amplifying the isolated nucleotides;
sequencing the amplified nucleotides;
mapping the sequenced nucleotides to a human reference genome;
identifying genetic variants from the human reference genome in the sequenced nucleotides;
generating a microsatellite instability (MSI) score; and
generating a report from the identified genetic variants and MSI score.

14. The system of claim 1, wherein the received state of an order indicates DNA processing of a specimen, the sequence of steps further comprising:
scraping a prepared FFPE slide to collect a sample of the specimen's tissue;
isolating DNA nucleotides from the sample;
amplifying the isolated nucleotides;
sequencing the amplified nucleotides;
mapping the sequenced nucleotides to a human reference genome;
identifying genetic variants from the human reference genome in the sequenced nucleotides;
identifying one or more copy number alterations; and
generating a report from the identified genetic variants and copy number alterations.

15. The system of claim 1, wherein the received state of an order indicates RNA processing of a specimen, the sequence of steps further comprising:
scraping a prepared FFPE slide to collect a sample of the specimen's tissue;
isolating RNA nucleotides from the sample;
amplifying the isolated nucleotides;
sequencing the amplified nucleotides;
mapping the sequenced nucleotides to a human reference genome;
measuring an abundance of at least one of the mapped nucleotides; and
identifying one or more fusions; and
generating a report from the measured abundance of the at least one of the mapped nucleotides and identified fusions.

16. A method for genomic test processing carried out using an order management engine and one or more order processing engines including a receiving engine and an execution engine, comprising:
at the receiving engine:
receiving a state of an order from the order management engine;
at the execution engine:
determining a sequence of steps to advance the received state of an order to a final state;
iteratively designating each step of the sequence of steps as completed before initiating the next step of the sequence of steps; and
advancing the state of the order to a final state when a last step of the sequence of steps is completed; and
at the broadcasting engine:
broadcasting the final state of the order to the order management engine, wherein the order management engine causes one of the one or more order processing engines to generate a next-generation sequencing report from the final state of the order.

17. The method of claim 16, wherein the received state of an order indicates DNA processing of a specimen, the method further comprising:
scraping a prepared FFPE slide to collect a sample of the specimen's tissue;
isolating DNA nucleotides from the sample;
amplifying the isolated nucleotides;
sequencing the amplified nucleotides;
mapping the sequenced nucleotides to a human reference genome;
identifying genetic variants from the human reference genome in the sequenced nucleotides; and
generating a report from the identified genetic variants.

18. The method of claim 16, wherein the received state of an order indicates RNA processing of a specimen, the method further comprising:
scraping a prepared FFPE slide to collect a sample of the specimen's tissue;
isolating RNA nucleotides from the sample;
amplifying the isolated nucleotides;
sequencing the amplified nucleotides;
mapping the sequenced nucleotides to a human reference genome;
measuring an abundance of at least one of the mapped nucleotides; and
generating a report from the measured abundance of the at least one of the mapped nucleotides.

19. The method of claim 16, further comprising:
receiving, by a first order processing engine, the state of an order indicating DNA processing of a specimen, comprising:
scraping a prepared FFPE slide to collect a first sample of the specimen's tissue;
isolating DNA nucleotides from the first sample;
amplifying the isolated DNA nucleotides;
sequencing the amplified nucleotides;
mapping the sequenced nucleotides to a human reference genome;
identifying genetic variants from the human reference genome in the sequenced nucleotides; and
generating a report from the identified genetic variants; and
receiving, by a second order processing engine, the state of the order indicating RNA processing of the specimen, comprising:
scraping a prepared FFPE slide to collect a second sample of the specimen's tissue;
isolating RNA nucleotides from the second sample;
amplifying the isolated RNA nucleotides;
sequencing the amplified nucleotides;
mapping the sequenced nucleotides to a human reference genome;
measuring an abundance of at least one of the mapped nucleotides; and
generating a report from the measured abundance of the at least one of the mapped nucleotides,
wherein the first and the second order processing engines operate concurrently.

20. The method of claim 17, wherein:
one or more of the following steps occur at a first geographic location:
scraping a prepared FFPE slide to collect a sample of the specimen's tissue;
isolating DNA nucleotides from the sample;
amplifying the isolated nucleotides; and
sequencing the amplified nucleotides; and
one or more of the following steps occur in a cloud-based architecture:
mapping the sequenced nucleotide to a human reference genome;
identifying genetic variants from the human reference genome in the sequenced nucleotides; and
generating a report from the identified genetic variants.

21. The method of claim 17, wherein:
one or more of the following steps occur at a first geographic location:
scraping a prepared FFPE slide to collect a sample of the specimen's tissue;
isolating RNA nucleotides from the sample;
amplifying the isolated nucleotides; and
sequencing the amplified nucleotides; and
one or more of the following steps occur at a second geographic location:
mapping the sequenced nucleotides to a human reference genome;
measuring an abundance of at least one of the mapped nucleotides; and
generating a report from the measured abundance of the at least one of the mapped nucleotides.

22. The method of claim 20, wherein the report from the identified genetic variants also comprises information based on a database of information of individuals with a health condition similar to that of a patient who was a source of the specimen.

23. The method of claim 21, wherein the report from the measured abundance of the at least one of the mapped nucleotides also comprises information based on a database of information of individuals with a health condition similar to that of a patient who was a source of the specimen.

24. The method of claim 16, wherein the genomic test includes tumor-normal sequencing, and wherein a first order processing engine receives the state of an order indicating DNA processing of a normal specimen and a second order processing engine receives the state of the order indicating DNA processing of a tumor specimen, further comprising:
at the first order processing engine:
scraping a prepared FFPE slide to collect a sample of the normal specimen's tissue;
isolating normal DNA nucleotides from the normal sample;
amplifying the isolated normal nucleotides;
sequencing the amplified normal nucleotides;
mapping the sequenced normal nucleotides to a human reference genome; and
identifying genetic variants of the normal sample from the human reference genome in the sequenced nucleotides;
at the second order processing engine:
scraping a prepared FFPE slide to collect a sample of the tumor specimen's tissue;
isolating tumor DNA nucleotides from the tumor sample;
amplifying the isolated tumor nucleotides;
sequencing the amplified tumor nucleotides;
mapping the sequenced tumor nucleotides to a human reference genome;
identifying genetic variants of the tumor sample from the human reference genome in the sequenced nucleotides; and
generating a report from the identified genetic variants of the tumor sample based at least in part on the identified genetic variants of the normal sample.

25. The method of claim 24, wherein:
one or more of the following steps occur at a first geographic location:
scraping a prepared FFPE slide to collect a sample of the normal specimen's tissue;
isolating normal DNA nucleotides from the normal sample;
amplifying the isolated normal nucleotides;
sequencing the amplified normal nucleotides;
scraping a prepared FFPE slide to collect a sample of the tumor specimen's tissue;
isolating tumor DNA nucleotides from the tumor sample;
amplifying the isolated tumor nucleotides;
sequencing the amplified tumor nucleotides; and
one or more of the following steps occur in a cloud-based architecture:
mapping the sequenced tumor nucleotides to a human reference genome;
mapping the sequenced normal nucleotides to a human reference genome;
identifying genetic variants of the tumor sample from the human reference genome in the sequenced nucleotides;
identifying genetic variants of the normal sample from the human reference genome in the sequenced nucleotides; and
generating a report from the identified genetic variants of the tumor sample based at least in part on the identified genetic variants of the normal sample.

26. The method of claim 16, wherein the received state of an order indicates DNA processing of a liquid biopsy specimen, the sequence of steps further comprising:
removing cells from the liquid biopsy specimen to collect a cell-free sample of the specimen;
isolating DNA nucleotides from the cell-free sample;
amplifying the isolated nucleotides;
sequencing the amplified nucleotides;
mapping the sequenced nucleotides to a human reference genome;
identifying genetic variants from the human reference genome in the sequenced nucleotides; and
generating a report from the identified genetic variants.

27. The method of claim 16, wherein the received state of an order indicates DNA processing of a specimen, the sequence of steps further comprising:
scraping a prepared FFPE slide to collect a sample of the specimen's tissue;
isolating DNA nucleotides from the sample;
amplifying the isolated nucleotides;
sequencing the amplified nucleotides;
mapping the sequenced nucleotides to a human reference genome;
identifying genetic variants from the human reference genome in the sequenced nucleotides;
generating a tumor mutational burden (TMB) score;
generating a microsatellite instability (MSI) score; and
generating a report from the identified genetic variants, TMB score, and MSI score.

28. The method of claim 16, wherein the received state of an order indicates DNA processing of a blood specimen, the sequence of steps further comprising:
extracting peripheral whole blood to collect a sample of the specimen;
isolating DNA nucleotides from the sample;
amplifying the isolated nucleotides;
sequencing the amplified nucleotides;
mapping the sequenced nucleotides to a human reference genome;
identifying genetic variants from the human reference genome in the sequenced nucleotides;
generating a microsatellite instability (MSI) score; and
generating a report from the identified genetic variants and MSI score.

29. The method of claim 16, wherein the received state of an order indicates DNA processing of a specimen, the sequence of steps further comprising:
scraping a prepared FFPE slide to collect a sample of the specimen's tissue;
isolating DNA nucleotides from the sample;
amplifying the isolated nucleotides;
sequencing the amplified nucleotides;
mapping the sequenced nucleotides to a human reference genome;
identifying genetic variants from the human reference genome in the sequenced nucleotides;
identifying one or more copy number alterations; and
generating a report from the identified genetic variants and copy number alterations.

30. The method of claim 16, wherein the received state of an order indicates RNA processing of a specimen, the sequence of steps further comprising:
scraping a prepared FFPE slide to collect a sample of the specimen's tissue;
isolating RNA nucleotides from the sample;
amplifying the isolated nucleotides;
sequencing the amplified nucleotides;
mapping the sequenced nucleotides to a human reference genome;
measuring an abundance of at least one of the mapped nucleotides; and identifying one or more fusions; and
generating a report from the measured abundance of the at least one of the mapped nucleotides and identified fusions.
